SEQUENCE LISTING (30 sequences)

SEQ ID NO: 1 - protein, 5 aa, synthetic construct
SYNVH

SEQ ID NO: 2 - protein, 17 aa, synthetic construct
TIYPGNLATSYNQKFKG

SEQ ID NO: 3 - protein, 6 aa, synthetic construct
SGTMDY

SEQ ID NO: 4 - protein, 12 aa, synthetic construct
RASSSISSTYLH

SEQ ID NO: 5 - protein, 7 aa, synthetic construct
NTQNLAS

SEQ ID NO: 6 - protein, 10 aa, synthetic construct
QQFGGYPLIT

SEQ ID NO: 7 - protein, 5 aa, synthetic construct
NYGMN

SEQ ID NO: 8 - protein, 17 aa, synthetic construct
WINTYTGEPTYAADFKR

SEQ ID NO: 9 - protein, 14 aa, synthetic construct
YPHYYGSSHWYFDV

SEQ ID NO: 10 - protein, 11 aa, synthetic construct
SASQDISNYLN

SEQ ID NO: 11 - protein, 7 aa, synthetic construct
FTSSLHS

SEQ ID NO: 12 - protein, 9 aa, synthetic construct
QQYSTVPWT

SEQ ID NO: 13 - protein, 115 aa, synthetic construct
QVQLVQSGAEVKKPGASVKVSCKASGYTFTSYNVHWVRQAPGQGLEWMGTIYPGNLATSYNQKFKGRVTLTADTSTSTVYMELSSLRSEDTAVYYCARSGTMDYWGQGTTVTVSS

SEQ ID NO: 14 - protein, 109 aa, synthetic construct
EIVLTQSPGTLSLSPGERATMTCRASSSISSTYLHWYQQKPGASPKLLIYNTQNLASGVPARFSGSGSGTSYTLTISRLEPEDFAVYYCQQFGGYPLITFGAGTKLELK

SEQ ID NO: 15 - protein, 123 aa, synthetic construct [sequence omitted]

SEQ ID NO: 16 - protein, 108 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 17 - protein, 15 aa, synthetic construct
GGGGSGGGGSGGGGS

SEQ ID NO: 18 - protein, 20 aa, synthetic construct
GGGGSGGGGSGGGGSGGGGS

SEQ ID NO: 19 - protein, 330 aa, synthetic construct
  VARIANT 249: note = T, W or S
  VARIANT 251: note = L or A
  VARIANT 290: note = Y or V
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLXCXVKGFYPSDIAVEWESNGQPENNYKTTPPVLDSDGSFFLXSKLTVDKSRWQQGNVFSCSVMHEALHNHYTQKSLSLSPGK

SEQ ID NO: 20 - protein, 107 aa, synthetic construct [sequence omitted]

SEQ ID NO: 21 - protein, 445 aa, synthetic construct
QVQLVQSGAEVKKPGASVKVSCKASGYTFTSYNVHWVRQAPGQGLEWMGTIYPGNLATSYNQKFKGRVTLTADTSTSTVYMELSSLRSEDTAVYYCARSGTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 216 aa, synthetic construct
EIVLTQSPGTLSLSPGERATMTCRASSSISSTYLHWYQQKPGASPKLLIYNTQNLASGVPARFSGSGSGTSYTLTISRLEPEDFAVYYCQQFGGYPLITFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 713 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLVQSGAEVKKPGASVKVSCKASGYTFTSYNVHWVRQAPGQGLEWMGTIYPGNLATSYNQKFKGRVTLTADTSTSTVYMELSSLRSEDTAVYYCARSGTMDYWGQGTTVTVSSGGGGSGGGGSGGGGSGGGGSEIVLTQSPGTLSLSPGERATMTCRASSSISSTYLHWYQQKPGASPKLLIYNTQNLASGVPARFSGSGSGTSYTLTISRLEPEDEAVYYCQQFGGYPLITFGAGTKLTAKR

SEQ ID NO: 26 - protein, 711 aa, synthetic construct
QVQLVQSGAEVKKPGASVKVSCKASGYTFTSYNVHWVRQAPGQGLEWMGTIYPGNLATSYNQKFKGRVTLTADTSTSTVYMELSSLRSEDTAVYYCARSGTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDEATYYCQQYSTVPWTFGQGTKLTAKR

SEQ ID NO: 27 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 28 - protein, 312 aa, Macaca fascicularis
MAFLVAPPMQFVYLLKTLCVFNMVFAKPGFSETVFSHRLSFTVLSAVGYFRWQKRPHLLPVSPLGRSMRWCLFLIWAQGLRQAPLASGMMTGTIETTGNISAKKGGSVILQCHLSSTMAQVTQVNWEQHDHSLLAIRNAELGWHIYPAFKDRVAPGPGLGLTLQSLTMNDTGEYFCTYHTYPDGTYRGRIFLEVLESSVAEHSARFQIPLLGAMAMMLVVICIAVIVVVVLARKKKSLRIHSVESGLQRKSTGQEEQIPSAPSPPGSCVQAEAAPAGLCGEQQGDDCAELHDYFNVLSYRSLGSCSFFTETG

SEQ ID NO: 29 - protein, 241 aa, Mus musculus
MHGWLLLVWVQGLIQAAFLATGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSVAQFQTAPLGGTMAAVLGLICLMVTGVTVLARKKSIRMHSIESGLGRTEAEPQEWNLRSLSSPGSPVQTQTAPAGPCGEQAEDDYADPQEYFNVLSYRSLESFIAVSKTG

SEQ ID NO: 30 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

(19) United States
(12) Patent Application Publication
(10) Pub. No.: US 2023/0348629 A1
(43) Pub. Date: Nov. 2, 2023

(54) BISPECIFIC MOLECULES BINDING TIGIT AND VEGF AND USES THEREOF

(71) Applicant: Beijing Mabworks Biotech Co., Ltd, Beijing (CN)

(72) Inventors: Jiangmei LI, Beijing (CN); Wenqi HU, Beijing (CN); Feng LI, Beijing (CN)

(21) Appl. No.: 18/307,215

(22) Filed: Apr. 26, 2023

(30) Foreign Application Priority Data
Apr. 28, 2022 (CN) 202210462385.8

(51) Int. Cl. C07K 16/46; C12N 15/63; A61P 35/00

(52) U.S. Cl. CPC C07K 16/468; C12N 15/63; A61P 35/00; C07K 2317/41; C07K 2317/565; C07K 2317/31; C07K 2317/732

(57) ABSTRACT

Disclosed is a bispecific molecule specifically binding TIGIT and VEGF, and its use in treatment of e.g., tumors.

### INCORPORATION BY REFERENCE

This application claims priority to Chinese Patent Application No. 202210462385.8 filed on Apr. 28, 2022.

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing XML labeled “55556-00097SequenceListingXML” which was created on Apr. 17, 2023 and is 33 bytes. The entire content of the sequence listing is incorporated herein by reference in its entirety.

### FIELD OF THE INVENTION

The present disclosure relates to a bispecific molecule binding TIGIT and VEGF, and the use of the molecule in treating diseases such as tumors.

### BACKGROUND OF THE INVENTION

### TIGIT and Immune Regulation

T cell immunoglobulin and ITIM domain (TIGIT), also referred to as V-set and immunoglobulin domain-containing protein 9 (VSIG9), V-set and transmembrane domain-containing protein 3 (Vstm3), or Washington University cell adhesion molecule (WUCAM), is an inhibitory immune checkpoint that belongs to the poliovirus receptor (PVR)-like protein family. It is a type I transmembrane protein, containing an extracellular immunoglobulin variable domain, a type I transmembrane domain and a short intracellular domain with one immunoreceptor tyrosine-based inhibitory motif (ITIM) and one immunoglobulin tyrosine tail (ITT)-like motif. The immunoglobulin variable domain shares sequence homology with other PVR-like proteins, including CD226 (DNAM-1), CD96, CD155, CD111, CD112, CD113 and PVRL4.

TIGIT is expressed on activated CD8⁺ T and CD4⁺ T cells, natural killer (NK) cells, regulatory T cells (Tregs), and follicular T helper cells in humans. It competes with CD226, a co-stimulatory receptor expressed on naive and resting T cells, over CD155 (PVR) binding, to counterbalance the costimulatory function of CD226, with its CD155 binding affinity much higher than that of CD226, wherein CD155 expression is found on tumor cells and antigen presenting cells. The relative amount of TIGIT-CD155 binding versus CD226-CD155 binding determines whether a T cell undergoes activation or anergy. The TIGIT-CD155 interaction may block T cell receptor (TCR) signaling, and inhibit pro-inflammatory cytokine production by CD4⁺ T cells (Shibuya K et al., (1999) Immunity 11:615-623; Lozano E et al., (2013) J Immunol 191:3673-3680). TIGIT expression is also found in about 20-90% resting NK cells, which level is increased following acute or chronic virus infection or oncogenesis. The engagement of TIGIT with CD155 initiates major inhibitory signaling in human NK cells via the ITT-like motif, and decreases these cells' reactions to tumor cells and capability to release interferon-α (Holder K A, Grant M D. (2020) Front Cell Infect Microbiol. 10:175; Stanietsky N et al., (2009) Proc Natl Acad Sci USA 106:17858-17863; Liu S et al., (2013) Cell Death Differ 20:456-464). Further, TIGIT⁺ Tregs are more immunosuppressive and may up-regulate TIM3 expression to further inhibit anti-tumor responses (Kurtulus S et al., (2015) J Clin Invest. 125(11):4053-4062).

Studies have shown TIGIT inhibits innate immunity and adaptive immunity through multiple ways. Anti-TIGIT antibodies have been developed and clinically tested for malignancy treatments. Etigilimab (OMP-313M32), of OncoMed Pharmaceuticals, was tested for its safety and pharmacokinetics in a Phase I, dose-escalation study (NCT031119428) as a single agent or in combination with nivolumab (anti-PD-1 mAb) in treatment of various advanced or metastatic solid malignancies, including colorectal cancer, endometrial cancer, and pancreatic cancer. The Phase Ia trial showed etigilimab was well tolerated at doses up to 20 mg/kg. Another antagonistic anti-TIGIT antibody, Tiragolumab, developed by Roche, was found effective against solid cancers, especially non-small cell lung cancer, when used in combination with the PD-L1 inhibitor atezolizumab. More anti-TIGIT antibodies, including BMS-986207 (Bristol-Myers Squibb), BGB-A1217 (BeiGene), and AB154 (Arcus biosciences), are being tested in clinical trials as a single agent or in combination with other anti-tumor agents for treating solid tumors such as multiple myeloma and melanoma (Chauvin J, Zarour H M., (2020) Journal for ImmunoTherapy of Cancer 8:e000957).

Studies further showed that the heavy chain constant region, e.g., the Fc region, of the anti-TIGIT antibodies may be required for the anti-tumor efficacy. The anti-TIGIT antibodies with the Fc regions may trigger macrophage and/or NK cell-mediated ADCP and/or ADCC against Tregs, while Treg clearance may promote CD8⁺ T cell infiltration in tumors (Argast G M et al., (2018) Cancer Res. 78(135):5627-27). The Fc-FcγR interaction may also activate myeloid cells, resulting in enhanced cytokine and chemokine production as well as robust perforin and granzyme B release (Han J H et al., (2020) Front Immunol. 11:573405).

### VEGF and Tumor Microenvironment

Vascular endothelial-derived growth factor (VEGF) is a family of homo-dimeric glycoproteins with pro-angiogenic activity, including VEGF-A, VEGF-B, VEGF-C, VEGF-D, VEGF-E and P1GF. VEGF, especially VEGF-A, plays an important role in angiogenesis and vascular permeability, and thus involved in physiological homeostasis and pathogenesis.

In diverse tumors, increased VEGF levels are associated with unfavorable clinical outcomes. In one aspect, VEGF binds to VEGFR1, VEGFR2 and/or VEGFR3 to phosphorylate tyrosine in the intracellular region of these VEGFRs, resulting in growth, proliferation and maturation of vascular endothelial cells and therefore formation of abnormal leaky blood vessels. In another aspect, VEGF suppresses anti-tumor immunity. In particular, VEGF inhibits dendritic cell maturation, leading to inactivation of cytotoxic T lymphocytes (CTLs), and activates regulatory T cells (Tregs), tumor associate macrophages (TAMs) and myeloid-derived suppressor cells (MDSCs), resulting in immune-suppressive tumor microenvironment (TME). Hypoxia in the tumor microenvironment may lead to recruitment of TAMs, Tregs and MDSCs directly or via VEGF upregulation, which may help tumor cells evade immune surveillance. VEGF may also increase PD-1 expression on CD8⁺ CTLs and Tregs in a VEGF2-dependent manner, and cooperate with IL-10 and prostaglandin E3 to induced Fas ligand expression in endothelial cells, causing exhaustion of CTLs but not Tregs.

Avastin® bevacizumab was approved by FDA in 2004 for treatment of metastatic colorectal cancer, and later for clinical treatment of e.g., non-squamous non-small-cell lung carcinoma, renal cell carcinoma, glioblastoma multiforme, ovarian cancer, and cervical carcinoma (Ferrara N, Adamis A P. (2016) 15(6):385-403). The VEGF blocking agent has also been used in combination with an anti-PD-1 antibody, and potent efficacy was observed against e.g., renal cell cancer, non-small cell lung cancer, and hepatocellular carcinoma. For example, according to the phase I clinical trial of KEYNOTE524, lenvatinib plus an anti-PD-1 antibody had long-term inhibitory effect on tumors, resulting in tumor shrinkage and a median follow-up duration of 10.6 months. Further, in IMbrave150, a global, multicenter, open-label, phase III randomized trial, the combination of atezolizumab and bevacizumab demonstrated statistically significant and clinically meaningful improvement in two primary endpoints, i.e., overall survival (OS) and progression-free survival (PFS).

### Bispecific Molecule Targeting TIGIT and VEGF

As VEGF and TIGIT are both present in the tumor microenvironment and function to modulate immune cell infiltration and Treg-associated immune-suppression, a bispecific molecule targeting the two molecules may be directed to and concentrated in tumor sites, and renders the TME less immune-suppressive by blocking two signaling pathways.

No such anti-VEGF/TIGIT molecule has been reported yet.

Citation or identification of any document in this application is not an admission that such document is available as prior art to the present invention.

### SUMMARY OF THE INVENTION

The inventors of the application have designed a bispecific molecule capable of binding TIGIT and VEGF simultaneously, which, compared to the monospecific prior art antibodies such as Bevacizumab and Tiragolumab, has i) comparable, if not higher, binding affinity/capability to human/monkey TIGIT and VEGF-A, ii) comparable, if not higher, inhibitory effect on VEGF-mediated cell proliferation, and TIGIT-PVR binding, and iii) comparable, if not higher, activity to induce T cell activation, and antibody-dependent cell-mediated cytotoxicity (ADCC) against TIGIT⁺ cells. The afucosylated bispecific molecule induces even enhanced ADCC. Further, the bispecific molecule has potent in vivo anti-tumor activity, and synergizes with an anti-PD-L1 antibody in tumor suppression.

In a first aspect, the disclosure provides a bispecific molecule, which may comprise a TIGIT binding domain and a VEGF binding domain. The TIGIT binding domain may be an anti-TIGIT antibody or an antigen binding fragment thereof. The VEGF binding domain may be an anti-VEGF antibody or an antigen binding fragment thereof. The TIGIT binding domain and the VEGF binding domain may be linked in e.g., Fab-Fab, Fv-Fv, scFv-Fab, scFv-Fv formats, as long as the two binding domains retain the TIGIT and VEGF binding capability and can block TIGIT-PVR and VEGF-VEGFR interactions. In certain embodiments, the VEGF may be VEGF-A.

The bispecific molecule of the disclosure, in one embodiment, may comprise one TIGIT binding domain, and one VEGF binding domain. The bispecific molecule of the disclosure, in one embodiment, may comprise two TIGIT binding domains, and two VEGF binding domains.

In one embodiment, the TIGIT binding domain may be a Fab or Fv fragment, and the VEGF binding domain may be a Fab or Fv fragment. In one embodiment, the TIGIT binding domain may be a scFv, and the VEGF binding domain may be a Fab or Fv fragment.

The bispecific molecule may further comprise a heavy chain constant region and/or a light chain constant region. The heavy chain constant region may be with FcR binding affinity, such that the bispecific molecule may trigger ADCC, ADCP and/or CDC against e.g., TIGIT⁺ target cells.

In one embodiment, the bispecific molecule of the disclosure may comprise:
i) a first polypeptide, containing, from N-terminus to C-terminus, an anti-TIGIT heavy chain variable region and a heavy chain constant region,
ii) a second polypeptide, containing an anti-TIGIT light chain variable region,
iii) a third polypeptide, containing, from N-terminus to C-terminus, an anti-VEGF heavy chain variable region, and a heavy chain constant region, and
iv) a fourth polypeptide, containing an anti-VEGF light chain variable region,
wherein the anti-TIGIT heavy chain variable region in the first polypeptide and the anti-TIGIT light chain variable region in the second polypeptide associate to form a TIGIT binding domain, the anti-VEGF heavy chain variable region in the third polypeptide and the anti-VEGF light chain variable region in the fourth polypeptide associate to form a VEGF binding domain, and the heavy chain constant region in the first polypeptide and the heavy chain constant region in the third polypeptide are associated together via e.g., the knobs-into-holes approach, the covalent bond(s) or the disulfide bond(s).

The anti-TIGIT heavy chain variable region in the first polypeptide may comprise a VH-CDR1, a VH-CDR2 and a VH-CDR3 that may comprise the amino acid sequences of SEQ ID NOs: 1, 2 and 3, respectively. The anti-TIGIT light chain variable region in the second polypeptide may comprise a VL-CDR1, a VL-CDR2 and a VL-CDR3 that may comprise the amino acid sequences of SEQ ID NOs: 4, 5 and 6, respectively. The anti-TIGIT heavy chain variable region in the first polypeptide may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 13, and the anti-TIGIT light chain variable region in the second polypeptide may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 14.

In one embodiment, the VEGF may be VEGF-A. The anti-VEGF heavy chain variable region in the third polypeptide may comprise a VH-CDR1, a VH-CDR2 and a VH-CDR3 that may comprise the amino acid sequences of SEQ ID NOs: 7, 8 and 9, respectively. The anti-VEGF light chain variable region in the fourth polypeptide may comprise a VL-CDR1, a VL-CDR2 and a VL-CDR3 that may comprise the amino acid sequences of SEQ ID NOs: 10, 11 and 12, respectively. The anti-VEGF heavy chain variable region in the third polypeptide may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 15, and the anti-VEGF light chain variable region in the fourth polypeptide may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 16.

The heavy chain constant region in the first polypeptide may be a hole variant, such as human IgG1 heavy chain constant region or a functional fragment thereof with the T366S/L368A/Y407V mutations. The heavy chain constant region in the first polypeptide may be a hole variant with FcR (e.g., FcγR) binding affinity, such as human IgG1 heavy chain constant region comprising the amino acid sequence of SEQ ID NO: 19 (X1=S, X2=A, X3=V). The heavy chain constant region in the third polypeptide may be a knob variant, such as human IgG1 heavy chain constant region or a functional fragment thereof with the T366W mutation. The heavy chain constant region in the third polypeptide may be a knob variant with FcR (e.g., FcγR) binding affinity, such as human IgG1 heavy chain constant region comprising the amino acid sequence of SEQ ID NO: 19 (X1=W, X2=L, X3=Y).

Alternatively, the heavy chain constant region in the first polypeptide may be a knob variant with FcR (e.g., FcγR) binding affinity, such as human IgG1 heavy chain constant region comprising the amino acid sequence of SEQ ID NO: 19 (X1=W, X2=L, X3=Y). The heavy chain constant region in the third polypeptide may be a hole variant with FcR (e.g., FcγR) binding affinity, such as human IgG1 heavy chain constant region comprising the amino acid sequence of SEQ ID NO: 19 (X1=S, X2=A, X3=V).

The second polypeptide and/or the fourth polypeptide may comprise a light chain constant region at the C-terminus, such as human κ or λ light chain constant region, comprising e.g., the amino acid sequence of SEQ ID NO: 20.

In one embodiment, the first, second, third and fourth polypeptides may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 21, 14, 23 and 16, respectively. In one embodiment, the first, second, third and fourth polypeptides may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 21, 22, 23 and 24, respectively.

In another embodiment, the bispecific molecule of the disclosure may comprise:
i) a first polypeptide, containing an anti-VEGF heavy chain variable region, a heavy chain constant region, an anti-TIGIT heavy chain variable region and an anti-TIGIT light chain variable region,
ii) a second polypeptide, containing an anti-VEGF light chain variable region,
iii) a third polypeptide, containing an anti-VEGF heavy chain variable region, a heavy chain constant region, an anti-TIGIT heavy chain variable region and an anti-TIGIT light chain variable region, and
iv) a fourth polypeptide, containing an anti-VEGF light chain variable region,
wherein the anti-VEGF heavy chain variable region in the first polypeptide and the anti-VEGF light chain variable region in the second polypeptide associate to form a VEGF binding domain, the anti-TIGIT heavy chain variable region and the anti-TIGIT light chain variable region in the first polypeptide associate to form a TIGIT binding domain, the anti-VEGF heavy chain variable region in the third polypeptide and the anti-VEGF light chain variable region in the fourth polypeptide associate to form a VEGF binding domain, the anti-TIGIT heavy chain variable region and the anti-TIGIT light chain variable region in the third polypeptide associate to form a TIGIT binding domain, and the heavy chain constant region in the first polypeptide and the heavy chain constant region in the third polypeptide are associated together via e.g., the knobs-into-holes approach, the covalent bond(s) or the disulfide bond(s).

The VEGF may be VEGF-A. The anti-VEGF heavy chain variable region in the first polypeptide may be same with or different from the anti-VEGF heavy chain variable region in the third polypeptide, and anti-VEGF light chain variable region in the second polypeptide may be same with or different from the anti-VEGF light chain variable region in the fourth polypeptide. The anti-VEGF heavy chain variable region in the first and/or third polypeptide(s) may comprise a VH-CDR1, a VH-CDR2 and a VH-CDR3 that may comprise the amino acid sequences of SEQ ID NOs: 7, 8 and 9, respectively. The anti-VEGF light chain variable region in the second and/or fourth polypeptide(s) may comprise a VL-CDR1, a VL-CDR2 and a VL-CDR3 that may comprise the amino acid sequences of SEQ ID NOs: 10, 11 and 12, respectively. The anti-VEGF heavy chain variable region in the first and/or third polypeptide(s) may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 15, and the anti-VEGF light chain variable region in the second and/or fourth polypeptide(s) may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 16.

The anti-TIGIT heavy chain variable region in the first polypeptide may be same with or different from the anti-TIGIT heavy chain variable region in the third polypeptide, and anti-TIGIT light chain variable region in the first polypeptide may be same with or different from the anti-TIGIT light chain variable region in the third polypeptide. The anti-TIGIT heavy chain variable region in the first and/or third polypeptide(s) may comprise a VH-CDR1, a VH-CDR2 and a VH-CDR3 that may comprise the amino acid sequences of SEQ ID NOs: 1, 2 and 3, respectively. The anti-TIGIT light chain variable region in the first and/or third polypeptide(s) may comprise a VL-CDR1, a VL-CDR2 and a VL-CDR3 that may comprise the amino acid sequences of SEQ ID NOs: 4, 5 and 6, respectively. The anti-TIGIT heavy chain variable region in the first and/or third polypeptide(s) may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 13, and the anti-TIGIT light chain variable region in the first and/or third polypeptide(s) may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 14.

The heavy chain constant region in the first and third polypeptides may be with FcR (e.g., FcγR) binding affinity, such as human IgG1 heavy chain constant region, or a functional fragment thereof. In one embodiment, the heavy chain constant region may comprise the amino acid sequence of SEQ ID NO: 19 (X1=T, X2=L, X3=Y). When the heavy chain constant region is linked at its C-terminus with a polypeptide such as an anti-TIGIT heavy chain variable region or an anti-TIGIT light chain variable region, the amino acid residue at the C-terminus, namely lysine (K), may be replaced with alanine (A) to enhance the connection stability.

The first polypeptide may comprise, from N-terminus to C-terminus, an anti-VEGF heavy chain variable region, a heavy chain constant region, an anti-TIGIT heavy chain variable region and an anti-TIGIT light chain variable region; or alternatively an anti-VEGF heavy chain variable region, a heavy chain constant region, an anti-TIGIT light chain variable region and an anti-TIGIT heavy chain variable region. The third polypeptide may comprise, from N-terminus to C-terminus, an anti-VEGF heavy chain variable region, a heavy chain constant region, an anti-TIGIT heavy chain variable region and an anti-TIGIT light chain variable region; or alternatively an anti-VEGF heavy chain variable region, a heavy chain constant region, an anti-TIGIT light chain variable region and an anti-TIGIT heavy chain variable region.

In the first and third polypeptides, the heavy chain constant region may be linked to the anti-TIGIT heavy or light chain variable region via a first linker. The first linker may be a peptide of about 5 to 30 amino acid residues. In one embodiment, the first linker may be a peptide of about 10 to 30 amino acid residues. In one embodiment, the first linker may be a peptide of about 10 to 20 amino acid residues. In one embodiment, the first linker may be a GS linker comprising e.g., the amino acid sequence of SEQ ID NOs: 17 or 18. In one embodiment, the first linker may be a GS linker comprising the amino acid sequence of SEQ ID NO: 17.

In the first and third polypeptides, the anti-TIGIT heavy chain variable region may be linked via a second linker to the anti-TIGIT light chain variable region. The second linker may be a peptide of about 5 to 30 amino acid residues. In one embodiment, the second linker may be a peptide of about 10 to 30 amino acid residues. In one embodiment, the second linker may be a peptide of about 10 to 20 amino acid residues. In one embodiment, the second linker may be a GS linker comprising e.g., the amino acid sequence of SEQ ID NOs: 17 or 18. In one embodiment, the second linker may be a GS linker comprising the amino acid sequence of SEQ ID NO: 18.

The second polypeptide and/or the fourth polypeptide may comprise a light chain constant region at the C-terminus, such as human κ or λ light chain constant region, comprising e.g., the amino acid sequence of SEQ ID NO: 20.

In one embodiment, the first and third polypeptides may comprise, from N-terminus to C-terminus, an anti-VEGF heavy chain variable region, a heavy chain constant region, an anti-TIGIT heavy chain variable region and an anti-TIGIT light chain variable region. In one embodiment, the first and third polypeptides may comprise, from N-terminus to C-terminus, an anti-VEGF heavy chain variable region, a heavy chain constant region, a first linker, an anti-TIGIT heavy chain variable region, a second linker, and an anti-TIGIT light chain variable region.

In one embodiment, the first, second, third and fourth polypeptides may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 25, 16, 25 and 16, respectively. In one embodiment, the first, second, third and fourth polypeptides may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 25, 24, 25 and 24, respectively.

The bispecific molecule of the disclosure may be afucosylated. For example, the bispecific molecule of the disclosure may be expressed in certain mammal cells to remove fucose from the oligosaccharides in the molecule. The cell lines for expressing afucosylated proteins such as the bispecific molecule of the disclosure include, but not limited to, a cell line lacking Slc35C1 gene, a cell line lacking FUT8 gene, a CHO variant cell line Lec13, a rat hybridoma cell line YB2/0, a cell line containing small interfering RNAs targeting FUT8, and a cell line co-expressing beta-1,4-N-acetyl-glucosamine transferase III and Golgi alpha-mannosidase II.

A nucleic acid molecule encoding the bispecific molecule or a functional fragment thereof of the disclosure, is also encompassed by the disclosure, as well as an expression vector that may comprise the nucleic acid molecule and a host cell that may comprise the expression vector or have the nucleic acid molecule integrated in its genome. A method for preparing the bispecific molecule or the functional fragment thereof of the disclosure using the host cell is also provided, that may comprise steps of (i) expressing the molecule or the functional fragment thereof in the host cell and (ii) isolating the molecule or the functional fragment thereof from the host cell or its cell culture.

A composition, e.g., a pharmaceutical composition, that may comprise the bispecific molecule or the functional fragment thereof, the nucleic acid molecule, the expression vector, or the host cell and a pharmaceutically acceptable carrier, is also provided.

In a second aspect, the disclosure provides a method for treating or alleviating a disease associated with TIGIT signaling and/or VEGF signaling in a subject in need thereof, comprising administering to the subject a therapeutically effective amount of the pharmaceutical composition of the disclosure.

In certain embodiments, the disease may be a tumor, such as a solid tumor, including, but not limited to, colorectal cancer, liver cancer, endometrial cancer, pancreatic cancer, non-small-cell carcinoma, multiple myeloma, melanoma, renal cell carcinoma, glioblastoma multiforme, ovarian cancer, hepatocellular carcinoma, and cervical carcinoma.

In one embodiment, the pharmaceutical composition of the disclosure may be administered with an agent inhibiting PD-1/PD-L1 signaling The agent inhibiting PD-1/PD-L1 signaling may be an anti-PD-1 antibody or an anti-PD-L1 antibody.

The disclosure further provides the use of the pharmaceutical composition of the disclosure in treating or alleviating a disease associated with TIGIT signaling and/or VEGF signaling. The disease includes, but not limited to, cancers and neovascular eye diseases. The tumor may be a solid tumor, such as colorectal cancer, liver cancer, endometrial cancer, pancreatic cancer, non-small-cell carcinoma, multiple myeloma, melanoma, renal cell carcinoma, glioblastoma multiforme, ovarian cancer, hepatocellular carcinoma, and cervical carcinoma. The neovascular eye disease may include, but not limited to, diabetic macular edema, diabetic retinopathy, retinal vein occlusion, age-related macular degeneration, and choroidal neovascularization. In certain embodiments, the disease may be atherosclerosis, sepsis, acute lung injury, or acute respiratory distress syndrome.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments as described, may best be understood in conjunction with the accompanying drawings.

FIG. 1 is the schematic diagram of structures of the bispecific molecules of the disclosure.

FIG. 2 shows the binding activity of the bispecific molecules to human VEGF-A (A), mouse VEGF-A (B), human VEGF-B (C) and human VEGF-C (D).

FIG. 3 shows the inhibitory effect of the bispecific molecules of the disclosure on HUVEC cell proliferation.

FIG. 4 shows the binding activity of the bispecific molecules of the disclosure to HEK293A/human TIGIT cells (A), HEK293A/monkey TIGIT cells (B) and HEK293A/mouse TIGIT cells (C).

FIG. 5 shows the effect of 50 ng/ml (A) and 50 μg/ml (B) free human VEGF-A molecules on the binding of the bispecific molecules of the disclosure to HEK293A/human TIGIT cells.

FIG. 6 shows the capability of the bispecific molecules of the disclosure to block PVR-TIGIT interaction.

FIG. 7 shows the capability of the bispecific molecules of the disclosure to induce secretion of IFN-γ (A) and IL-2 (B) by T cells.

FIG. 8 shows the capability of the bispecific molecules of the disclosure to trigger ADCC against HEK293A/human TIGIT cells by NK92 cells (A) or PBMCs (B).

FIG. 9 shows the binding affinity of the bispecific molecules of the disclosure to human TIGIT (A, B, C) and human VEGF-A (D, E, F).

FIG. 10 shows the binding capability of MBS310-6 (A, B) and MBS310-7 (C, D) to human TIGIT and human VEGF-A simultaneously.

FIG. 11 shows the capability of the afucosylated bispecific molecules of the disclosure to induce ADCC against HEK293A/human TIGIT cells by NK92 cells (A) and to enhance NK92 cell activation (B).

FIG. 12 shows the average tumor sizes of the tumor-bearing mice treated by 70E11VH2VL4-AF, MBS310-6-AF, atezolizumab, or atezolizumab in combination with MBS310-6-AF

### DETAILED DESCRIPTION OF THE INVENTION

To ensure that the present disclosure may be more readily understood, certain terms are first defined. Additional definitions are set forth throughout the detailed description.

The term “TIGIT” refers to T cell immunoglobulin and ITIM domain. The term may comprise variants, isoforms, homologs, orthologs and paralogs. For example, a molecule such as an antibody specific for a human TIGIT protein may, in certain cases, cross-react with a TIGIT protein from a species other than human, such as monkey. In other embodiments, a molecule such as an antibody specific for a human TIGIT protein may be completely specific for the human TIGIT protein and exhibit no cross-reactivity to other species or of other types, or may cross-react with TIGIT from certain other species but not all other species.

The term “human TIGIT” refers to a TIGIT protein having an amino acid sequence from a human, such as the amino acid sequence of SEQ ID NO: 27. The terms “monkey or rhesus TIGIT” and “mouse TIGIT” refer to monkey and mouse TIGIT sequences, respectively, e.g., those with the amino acid sequences of SEQ ID NOs: 28 and 29, respectively.

The term “VEGF” refers to vascular endothelial-derived growth factor, including VEGF-A, VEGF-B, VEGF-C, VEGF-D, VEGF-E and P1GF. The term “human VEGF-A” refers to a VEGF-A protein having an amino acid sequence from human. Due to alternative mRNA splicing, VEGF-A contains several splice variants, including VEGF165.

The term “antibody” as referred to herein includes IgG, IgA, IgD, IgE and IgM whole antibodies and any antigen binding fragment (i.e., “antigen-binding portion”) or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The “functional fragment” of a heavy chain constant region refers to the part of the constant region that retains certain activity such as the binding affinity to FcRs and/or the complement system component(s).

The “knob variant” of a heavy chain constant region, or a heavy chain constant region with “knob mutation(s)” refers to a heavy chain constant region used in the knobs-into-holes technology whose CH3 domains are engineered to create a “knob”. Similarly, the “hole variant” of a heavy chain constant region, or a heavy chain constant region with “hole mutation(s)” refers to a heavy chain constant region used in the knobs-into-holes technology whose CH3 domains are engineered to create a “hole”.

The term “antigen binding fragment” or “antigen-binding portion” of an antibody (or simply “antibody portion”), as used herein, refers to one or more fragments of an antibody that retain the ability to specifically bind to an antigen (e.g., a TIGIT or VEGF protein). It has been shown that the antigen-binding function of an antibody can be performed by fragments of a full-length antibody. Examples of binding fragments encompassed within the term “antigen binding fragment” or “antigen-binding portion” of an antibody include (i) a Fab fragment, a monovalent fragment consisting of the V_{L} V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen binding fragment” of an antibody. These antibody fragments are obtained using conventional techniques known to those with skill in the art, and the fragments are screened for utility in the same manner as are intact antibodies.

The term “FcR” or “Fc receptor” refers to a protein expressed on the surface of certain immune cells such as B lymphocytes, natural killer cells, and macrophages, which recognizes the Fc fragment of antibodies that are attached to cells or pathogens, and stimulates phagocytic or cytotoxic cells to destroy pathogens or target cells by e.g., antibody-mediated phagocytosis or antibody-dependent cell-mediated cytotoxicity. The FcR includes, FcαR, FcεR and FcγR, and the FcγR belongs to the immunoglobulin superfamily and is the most important Fc receptor for inducing phagocytosis of microbes, including FcγRI (CD64), FcγRIIA (CD32A), FcγRIIB (CD32B), and FcγRIIIA (CD16A).

A “bispecific” molecule, as used herein, specifically binds two target molecules, or two different epitopes in a same target molecule. The bispecific antibody of the disclosure specifically binds VEGF and TIGIT. In contrast, a “monospecific” molecule specifically binds a certain target molecule, especially a certain epitope in the target molecule, such as a monospecific anti-TIGIT antibody, or a monospecific anti-VEGF antibody. The “functional fragment” of a bispecific molecule refers to the part of the bispecific molecule that retains the binding affinity to target(s) (TIGIT and VEGF-A), optionally the binding affinity to FcRs, and other required characteristics.

The term “half antibody” or “half-antibody” refers to one half of an antibody which comprises e.g., a heavy chain and a light chain.

The percent “sequence identity” as used herein in the context of two or more nucleic acids or polypeptides, refers to two or more sequences or subsequences that are the same or have a specified percentage of nucleotides or amino acid residues that are the same, when compared and aligned (introducing gaps, if necessary) for maximum correspondence, considering or not considering conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection.

The term “EC₅₀”, also known as half maximal effective concentration, refers to the concentration of a molecule which induces a response halfway between the baseline and maximum after a specified exposure time.

The term “IC₅₀”, also known as half maximal inhibitory concentration, refers to the concentration of a molecule which inhibits a specific biological or biochemical function by 50% relative to the absence of the antibody.

The term “subject” includes any human or nonhuman animal The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the molecule or the functional fragment thereof of the present disclosure sufficient to prevent or ameliorate the symptoms associated with a disease or condition (such as a cancers) and/or lessen the severity of the disease or condition. A therapeutically effective amount is understood to be in context to the condition being treated, where the actual effective amount is readily discerned by those of skill in the art.

The term “ADCC” or “antibody dependent cell-mediated cytotoxicity” refers to a mechanism of cell mediated immunity where the Fc portion of an antibody-like molecule binds to the Fc receptors of immune effector cells (mainly natural killer cells), resulting in the release of cytotoxic granules from the immune effector cells, which cause the death of the antibody-like molecule-coated cells.

The term “ADCP” or “antibody dependent cellular phagocytosis” refers to a mechanism of cell mediated immunity where the Fc portion of an antibody-like molecule binds to the Fc receptors on phagocytes (i.e., macrophages, granulocytes and dendritic cells) to induce phagocytosis of cells bound by the antibody-like molecules.

The term “CDC” or “complement-dependent cytotoxicity” refers to a mechanism of antibody mediated immunity where an antibody-like molecule binds to the complement component C1q and activates the classical complement cascade, leading to the formation of a membrane attack complex (MAC) on the cell surface bound by the antibody-like molecules and subsequent cell lysis.

Various aspects of the disclosure are described in further detail in the following subsections.

### Bispecific Molecules

The inventors of the application designed a bispecific molecule which can bind TIGIT and VEGF simultaneously. When the bispecific molecule contains an anti-VEGF scFv linked to the C-terminus of the heavy chain of an IgG anti-TIGIT antibody, its binding affinity to VEGF is significantly attenuated. However, the bispecific molecule has high binding affinity to both TIGIT and VEGF when an anti-TIGIT scFv is linked to the C-terminus of the heavy chain of an IgG anti-VEGF antibody, or alternatively when an anti-TIGIT half antibody is in combination with an anti-VEGF half antibody.

Two exemplary bispecific molecules of the disclosure, compared to the monospecific prior art antibodies such as Bevacizumab and Tiragolumab, show i) comparable, if not higher, binding affinity/capability to human/monkey TIGIT and VEGF-A, ii) comparable, if not higher, inhibitory effect on VEGF-mediated cell proliferation, and TIGIT-PVR binding, and iii) comparable, if not higher, activity to induce T cell activation, and antibody-dependent cell-mediated cytotoxicity (ADCC) against TIGIT⁺ cells. The afucosylated bispecific molecules of the disclosure induce even higher ADCC. Further, the exemplary bispecific molecules of the disclosure have potent in vivo anti-tumor activity, and synergize with an anti-PD-L1 antibody in tumor suppression.

The anti-TIGIT antibody 70E11VH2VL4 as contained in the bispecific molecule of the disclosure is a humanized antibody or an antigen binding fragment thereof.

The heavy chain variable region CDRs and light chain variable region CDRs of the monospecific antibodies or antigen binding fragments thereof used herein have been defined by the Kabat numbering system. However, as is well known in the art, CDRs can also be determined by other systems such as Chothia, and IMGT, AbM, or Contact numbering system/method, based on heavy chain/light chain variable region sequences.

The bispecific molecule of the disclosure may contain a TIGIT binding domain and a VEGF binding domain. The VEGF may be VEGF-A.

In addition to the binding affinity and specificity to TIGIT and VEGF, the bispecific molecule of the disclosure may further contain binding affinity to e.g., FcRs. Thus, as used herein, “bispecific molecule” includes molecules that have three or more binding specificities, and may, in certain embodiments, be referred to as “multi-specific molecule”.

The bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv)₂ construct. Intermediate-sized bispecific molecules include two different F(ab) fragments linked by a peptidyl linker, and one F(ab) fragment linked to a scFv via a peptidyl linker. Bispecific molecules of these and other formats can be prepared by genetic engineering, somatic hybridization, or chemical synthesis methods.

As both VEGF and TIGIT are expressed in the tumor microenvironment and function to modulate immune cell infiltration and Treg-mediated immune-suppression, the bispecific molecule of the disclosure may be directed to and concentrated in the tumor sites through binding to VEGF (e.g., VEGF-A) in the TME and block two signaling pathways to render the TME less immune-suppressive.

In the bispecific molecule of the disclosure, the TIGIT binding domain may be an anti-TIGIT antibody or an antigen binding fragment thereof. The VEGF binding domain may be an anti-VEGF antibody or an antigen binding fragment thereof. The TIGIT binding domain and the VEGF binding domain may be linked in e.g., Fab-Fab, Fv-Fv, scFv-Fab, scFv-Fv formats, as long as the two binding domains retain the TIGIT and VEGF binding capability and can block VEGF-VEGFR and TIGIT-PVR interactions. In certain embodiments, the VEGF may be VEGF-A.

The bispecific molecule of the disclosure, in one embodiment, may comprise one TIGIT binding domain, and one VEGF binding domain. The bispecific molecule of the disclosure, in one embodiment, may comprise two TIGIT binding domains, and two VEGF binding domains In one embodiment, the TIGIT binding domain may be a Fab or Fv fragment, and the VEGF binding domain may be a Fab or Fv fragment. In one embodiment, the TIGIT binding domain may be a scFv, and the VEGF binding domain may be a Fab or Fv fragment.

The bispecific molecule may further comprise a heavy chain constant region and/or a light chain constant region. The heavy chain constant region may be with FcR binding affinity, such that the bispecific molecule may trigger ADCC, ADCP and/or CDC against e.g., TIGIT⁺ target cells.

The bispecific molecule of the disclosure may comprise:
i) a first polypeptide, containing, from N-terminus to C-terminus, an anti-TIGIT heavy chain variable region and a heavy chain constant region,
ii) a second polypeptide, containing an anti-TIGIT light chain variable region,
iii) a third polypeptide, containing, from N-terminus to C-terminus, an anti-VEGF heavy chain variable region, and a heavy chain constant region, and
iv) a fourth polypeptide, containing an anti-VEGF light chain variable region,
wherein the anti-TIGIT heavy chain variable region in the first polypeptide and the anti-TIGIT light chain variable region in the second polypeptide associate to form a TIGIT binding domain, the anti-VEGF heavy chain variable region in the third polypeptide and the anti-VEGF light chain variable region in the fourth polypeptide associate to form a VEGF binding domain, and the heavy chain constant region in the first polypeptide and the heavy chain constant region in the third polypeptide are associated together via e.g., the knobs-into-holes approach, the covalent bond(s) or the disulfide bond(s).

The anti-TIGIT heavy chain variable region and the anti-TIGIT light chain variable region may comprise a VH-CDR1, a VH-CDR2, a VH-CDR3, a VL-CDR1, a VL-CDR2 and a VL-CDR3 that may comprise the amino acid sequences of SEQ ID NOs: 1, 2, 3, 4, 5 and 6, respectively. The anti-TIGIT heavy chain variable region and the anti-TIGIT light chain variable region may comprise the amino acid sequences of SEQ ID NOs: 13 and 14, respectively.

The VEGF may be VEGF-A. The anti-VEGF heavy chain variable region and the anti-VEGF light chain variable region may comprise a VH-CDR1, a VH-CDR2, a VH-CDR3, a VL-CDR1, a VL-CDR2 and a VL-CDR3 that may comprise the amino acid sequences of SEQ ID NOs: 7, 8, 9, 10, 11 and 12, respectively. The anti-VEGF heavy chain variable region and the anti-VEGF light chain variable region may comprise the amino acid sequences of SEQ ID NOs: 15 and 16, respectively.

With regard to the heavy chain constant regions in the first and third polypeptides, one may be a hole variant with mutations forming a hole in structure, and the other may be a knob variant with mutations forming a knob in structure.

The second polypeptide and/or the fourth polypeptide may comprise a light chain constant region at the C-terminus, such as human κ or λ light chain constant region.

In one embodiment, the first, second, third and fourth polypeptides may comprise amino acid sequences of SEQ ID NOs: 21, 14, 23 and 16, respectively. In one embodiment, the first, second, third and fourth polypeptides may comprise amino acid sequences of SEQ ID NOs: 21, 22, 23 and 24, respectively.

In another embodiment, the bispecific molecule of the disclosure may comprise:
i) a first polypeptide, containing an anti-VEGF heavy chain variable region, a heavy chain constant region, an anti-TIGIT heavy chain variable region and an anti-TIGIT light chain variable region,
ii) a second polypeptide, containing an anti-VEGF light chain variable region,
iii) a third polypeptide, containing an anti-VEGF heavy chain variable region, a heavy chain constant region, an anti-TIGIT heavy chain variable region and an anti-TIGIT light chain variable region, and
iv) a fourth polypeptide, containing an anti-VEGF light chain variable region,
wherein the anti-VEGF heavy chain variable region in the first polypeptide and the anti-VEGF light chain variable region in the second polypeptide associate to form a VEGF binding domain, the anti-TIGIT heavy chain variable region and the anti-TIGIT light chain variable region in the first polypeptide associate to form a TIGIT binding domain, the anti-VEGF heavy chain variable region in the third polypeptide and the anti-VEGF light chain variable region in the fourth polypeptide associate to form a VEGF binding domain, the anti-TIGIT heavy chain variable region and the anti-TIGIT light chain variable region in the third polypeptide associate to form a TIGIT binding domain, and the heavy chain constant region in the first polypeptide and the heavy chain constant region in the third polypeptide are associated together via e.g., the knobs-into-holes approach, the covalent bond(s) or the disulfide bond(s).

The VEGF may be VEGF-A. The anti-VEGF heavy chain variable region in the first polypeptide may be same with or different from the anti-VEGF heavy chain variable region in the third polypeptide, and anti-VEGF light chain variable region in the second polypeptide may be same with or different from the anti-VEGF light chain variable region in the fourth polypeptide. The anti-VEGF heavy chain variable region and the anti-VEGF light chain variable region may comprise a VH-CDR1, a VH-CDR2, a VH-CDR3, a VL-CDR1, a VL-CDR2 and a VL-CDR3 that may comprise the amino acid sequences of SEQ ID NOs: 7, 8, 9, 10, 11 and 12, respectively. The anti-VEGF heavy chain variable region and the anti-VEGF light chain variable region may comprise the amino acid sequences of SEQ ID NOs: 15 and 16, respectively.

The anti-TIGIT heavy chain variable region in the first polypeptide may be same with or different from the anti-TIGIT heavy chain variable region in the third polypeptide, and anti-TIGIT light chain variable region in the first polypeptide may be same with or different from the anti-TIGIT light chain variable region in the third polypeptide. The anti-TIGIT heavy chain variable region and the anti-TIGIT light chain variable region may comprise a VH-CDR1, a VH-CDR2, a VH-CDR3, a VL-CDR1, a VL-CDR2 and a VL-CDR3 that may comprise the amino acid sequences of SEQ ID NOs: 1, 2, 3, 4, 5 and 6, respectively. The anti-TIGIT heavy chain variable region and the anti-TIGIT light chain variable region may comprise the amino acid sequences of SEQ ID NOs: 13 and 14, respectively.

The heavy chain constant region in the first and third polypeptides may be with FcR (e.g., FcγR) binding affinity. When the heavy chain constant region is linked at its C-terminus with a polypeptide such as an anti-TIGIT heavy chain variable region or an anti-TIGIT light chain variable region, the amino acid residue at the C-terminus, namely lysine (K), may be replaced with alanine (A) to enhance the connection stability.

The heavy chain constant region may be linked to the anti-TIGIT heavy or light chain variable region via a first linker in the first and third polypeptides.

In the first and third polypeptides, the anti-TIGIT heavy chain variable region may be linked via a second linker to the anti-TIGIT light chain variable region.

The second polypeptide and/or the fourth polypeptide may comprise a light chain constant region at the C-terminus, such as human κ or λ light chain constant region.

In one embodiment, the first, second, third and fourth polypeptides may comprise amino acid sequences of SEQ ID NOs: 25, 16, 25 and 16, respectively. In one embodiment, the first, second, third and fourth polypeptides may comprise amino acid sequences of SEQ ID NOs: 25, 24, 25 and 24, respectively.

### Linkers

The linker, including the first linker and the second linker of the disclosure, may be made up of amino acids linked together by peptide bonds, preferably from 5 to 30 amino acids linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines, particularly poly(Gly-Ala), and polyalanines. One exemplary linker as used may comprise the amino acid sequence of SEQ ID NOs: 17 or 18.

The linker may also be a non-peptide linker. For example, alkyl linkers such as —NH—, —(CH₂)s—C(O)—, wherein s=2-20 can be used. These alkyl linkers may further be substituted by any non-sterically hindering group such as lower alkyl (e.g., C₁₋₄) lower acyl, halogen (e.g., CI, Br), CN, NH₂, phenyl, etc.

### Conservative Modifications

The bispecific molecule of the disclosure may comprise a heavy and/or light chain variable region sequences or CDR1, CDR2 and CDR3 sequences with one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J. Biol. Chem. 272:26864-26870; Hall et al., (1992) J. Immunol. 149:1605-12; Kelley and O'Connell (1993) Biochem. 32:6862-35; Adib-Conquy et al., (1998) Int. Immunol. 10:341-6 and Beers et al., (2000) Clin. Can. Res. 6:2835-43.

As used herein, the term “conservative sequence modification” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

### Engineered Bispecific Molecules

The bispecific molecule of the disclosure can be prepared using a bispecific molecule having one or more of the V_{H}/V_{L} sequences of the present disclosure, as starting material to engineer a modified bispecific molecule. A bispecific molecule can be engineered by modifying one or more residues within one or both variable regions (i.e., V_{H} and/or V_{L}), for example within one or more CDR regions and/or within one or more framework regions. Additionally or alternatively, a bispecific molecule can be engineered by modifying residues within the constant region(s), for example to alter the effector function(s) of the antibody.

In certain embodiments, CDR grafting can be used to engineer the variable regions. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Therefore, the heavy and/or light chain variable region(s) in the bispecific molecules of the disclosure may contain the VH-CDR1, VH-CDR2, and VH-CDR3, and/or the VL-CDR1, VL-CDR2 and VL-CDR3, but different framework regions.

The framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the bispecific molecule of the disclosure are those that are structurally similar to the framework sequences used by the antibodies of the disclosure. The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Engineered antibodies of the disclosure include those in which modifications have been made to framework residues within V_{H} and/or V_{L}, e.g., to reduce the potential immunogenicity. One approach is to “back mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, the bispecific molecule of the disclosure can be engineered to include modifications within the Fc region, typically to alter one or more functional properties, such as serum half-life, complement fixation, Fc receptor binding, and/or antigen-dependent cellular cytotoxicity. Furthermore, the bispecific molecule of the disclosure can be chemically modified (e.g., one or more chemical moieties can be attached to the molecule) or be modified to alter its glycosylation, again to alter one or more functional properties.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of an antibody is mutated to decrease the biological half-life of the bispecific molecule. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of the bispecific molecule is modified. For example, a de-glycosylated molecule can be made (i.e., the molecule lacks glycosylation). Glycosylation can be altered to, for example, increase the affinity of the bispecific molecule for antigen. Such carbohydrate modifications can be accomplished by, for example, altering one or more sites of glycosylation. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, a bispecific molecule can be made that has an altered type of glycosylation, such as a hypofucosylated molecule having reduced amounts of fucosyl residues or a molecule having increased bisecting GlcNac structures. Such altered glycosylation patterns have been demonstrated to increase or reduce the ADCC ability of the bispecific molecule. Such carbohydrate modifications can be accomplished by, for example, expressing the bispecific molecule in a host cell with altered glycosylation machinery. Cells with altered glycosylation machinery have been described in the art and can be used as host cells in which to express the bispecific molecule of the disclosure to thereby produce a molecule with altered glycosylation. For example, the cell lines Ms704, Ms705, and Ms709 lack the fucosyltransferase gene, FUT8 (α(1,6)-fucosyltransferase), such that molecule expressed in the Ms704, Ms705, and Ms709 cell lines lacks fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that molecule as expressed in such a cell line exhibits hypofucosylation by reducing or eliminating the α-1,6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662).

Another modification of the bispecific molecule herein is pegylation. A bispecific molecule can be pegylated to, for example, increase the biological (e.g., serum) half-life. To pegylate a molecule, the molecule typically is reacted with polyethylene glycol (PEG), such as a reactive ester or aldehyde derivative of PEG, under conditions in which one or more PEG groups become attached to the molecule. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. Methods for pegylating proteins are known in the art and can be applied to the antibodies of the disclosure. See, e.g., EP 0 154 316 and EP 0 401 384.

### Nucleic Acid Molecules

In another aspect, the disclosure provides a nucleic acid molecule that encodes the bispecific molecule or a functional fragment thereof, of the disclosure, including those encoding the polypeptides constituting the bispecific molecule or functional fragment thereof of the disclosure.

The nucleic acid molecule can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

The nucleic acid molecule of the disclosure can be obtained using standard molecular biology techniques. Preferred nucleic acids molecules of the disclosure include those encoding the V_{H} and/or V_{L} sequences of the anti-VEGF or anti-TIGIT monoclonal antibody or the CDRs. Once DNA fragments encoding V_{H} and/or V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker.

For the bispecific molecule of the disclosure, nucleic acid sequences encoding the anti-VEGF antibodies' CDRs, VH and VL, the anti-TIGIT antibodies' VH and VL, and linkers are firstly synthesized, and then combined according to the structures of required bispecific molecules. For example, the DNA sequences coding for the anti-VEGF heavy chain variable region, the heavy chain constant region, the anti-TIGIT heavy chain variable region, the linker, and the anti-TIGITI light chain variable region can be “operatively” linked.

### Generation of Bispecific Molecules

The bispecific molecule of the disclosure may be produced by i) inserting the nucleotide sequences encoding polypeptides of the bispecific molecule into one or more expression vectors which are operatively linked to regulatory sequences transcription and translation that control transcription or translation; (ii) transducing or transfecting host cells with expression vectors; and (iii) expressing polypeptides to form the bispecific molecule of the disclosure.

The term “regulatory sequence” is intended to include promoters, enhancers and other expression control elements (e.g., polyadenylation signals) that control the transcription or translation of the antibody genes. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The expression vector can encode a signal peptide that facilitates secretion of the polypeptide chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the polypeptide chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

The expression vector(s) can be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the bispecific molecule of the disclosure in either prokaryotic or eukaryotic host cells, expression of the bispecific molecule in eukaryotic cells, and most preferably mammalian host cells, is the most preferred because such eukaryotic cells, and in particular mammalian cells, are more likely than prokaryotic cells to assemble and secrete a properly folded and immunologically active molecule.

The expression vectors that can be used in the present application include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

Preferred mammalian host cells for expressing the bispecific molecule of the disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding the bispecific molecule are introduced into mammalian host cells, the bispecific molecule is produced by culturing the host cells for a period of time sufficient to allow for expression of the bispecific molecule in the host cells or, more preferably, secretion of the bispecific molecule into the culture medium in which the host cells are grown. The bispecific molecule can be recovered from the culture medium using standard protein purification methods.

### Pharmaceutical Compositions

In another aspect, the present disclosure provides a pharmaceutical composition which may comprise the bispecific molecule or functional fragment thereof, the nucleic acid molecule, the expression vector, or the host cell, of the disclosure, formulated together with a pharmaceutically acceptable carrier. The pharmaceutical composition may optionally contain one or more additional pharmaceutically active ingredients, such as an anti-tumor antibody, or alternatively a non-antibody anti-tumor agent. The pharmaceutical composition of the disclosure may be used in combination with an additional anti-tumor agent.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a micro-emulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about 99% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

The administration of the bispecific molecule of the disclosure may be determined by physicians depending on a subject's e.g., sex, age, medical history and etc.

A “therapeutically effective dosage” of the bispecific molecule of the disclosure, may result in a decrease in severity of disease symptoms, or an increase in frequency and duration of disease symptom-free periods. For example, for the treatment of tumor-bearing subjects, a “therapeutically effective dosage” preferably reduces tumor size by at least about 20%, more preferably by at least about 40%, even more preferably by at least about 60%, and still more preferably by at least about 80%, or even eliminate tumors, relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Pharmaceutical compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the antibodies of the disclosure can be formulated to ensure proper distribution in vivo. For example, to ensure that the therapeutic antibody or antigen-binding portion thereof of the disclosure cross the blood-brain barrier, they can be formulated in liposomes, which may additionally comprise targeting moieties to enhance selective transport to specific cells or organs.

### Uses and Methods

The pharmaceutical composition of the disclosure has multiple in vitro and in vivo applications. For example, the composition may be used to treat or alleviate diseases associated with TIGIT signaling and/or VEGF signaling.

The pharmaceutical composition of the disclosure may be used to treat or alleviate tumors. The tumor may be a solid tumor, including, but not limited to, colorectal cancer, liver cancer, endometrial cancer, pancreatic cancer, non-small-cell carcinoma, multiple myeloma, melanoma, renal cell carcinoma, glioblastoma multiforme, ovarian cancer, hepatocellular carcinoma, and cervical carcinoma.

The pharmaceutical composition of the disclosure may be used to treat or alleviate other diseases associated with the TIGIT signaling and/or VEGF signaling, including, but not limited to, neovascular eye disease, atherosclerosis, sepsis, acute lung injury, and acute respiratory distress syndrome. The neovascular eye disease may include, but not limited to, diabetic macular edema, diabetic retinopathy, retinal vein occlusion, age-related macular degeneration, and choroidal neovascularization.

The pharmaceutical composition of the disclosure may be used to active T cells.

The disclosure provides methods of combination therapy in which the pharmaceutical composition of the present disclosure is co-administered with one or more additional antibodies or non-antibody agents, e.g., anti-PD-1 antibodies, and anti-PD-L1 antibodies, for treatment or alleviation of certain diseases.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Construction of Cell Lines Stably Expressing TIGIT or PVR

Cell lines stably expressing human TIGIT, monkey TIGIT, mouse TIGIT, or human PVR were constructed using HEK293A cells. Briefly, sequences encoding human TIGIT, monkey TIGIT, mouse TIGIT, and human PVR (amino acid sequences set forth in SEQ ID NOs: 27-30, respectively) were synthesized, and then subcloned into pLV-EGFP(2A)-Puro vectors (Beijing Inovogen, China). Lentiviruses were generated in HEK293T cells (Cobioer, NJ, China) by cotransfection of the resultant expression vectors (i.e., pLV-EGFP(2A)-Puro-TIGIT or pLV-EGFP(2A)-Puro-PVR), psPAX and pMD2.G plasmids, according to the instruction in Lipofectamine 3000 kit (Thermo Fisher Scientific, USA). Three days post cotransfection, the lentiviruses were harvested from the HEK293T cell culture supernatants, and then used to infect HEK293A cells (Cobioer, NJ, China) to generate HEK293A/human TIGIT cells, HEK293A/monkey TIGIT cells, and HEK293A/mouse TIGIT cell, or alternatively to infect A549 cells (Cobioer, NJ, China) to generate A549/human PVR cells. These HEK293A cells and A549 cells were cultured in DMEM (Cat #:SH30022.01, Gibco, USA) containing 10% FBS (Cat #:FND500, Excell, China) and 0.2 μg/ml puromycin (Cat #:A11138-03, Gibco) for 7 days. The expressions of human and monkey TIGIT were confirmed by FACS using commercially available anti-TIGIT antibody (PE anti-human TIGIT Antibody, Cat #:357503, Biolegend, USA). Similarly, the expressions of mouse TIGIT and human PVR were measured by FACS using the PE-anti-mouse TIGIT antibody (Cat #:622205, Biolegend, USA), and PE-anti-human PVR antibody (Cat #:566718, BD, USA), respectively.

### Example 2. Construction and Expression of Exemplary Anti-VEGF/TIGIT Bispecific Antibodies

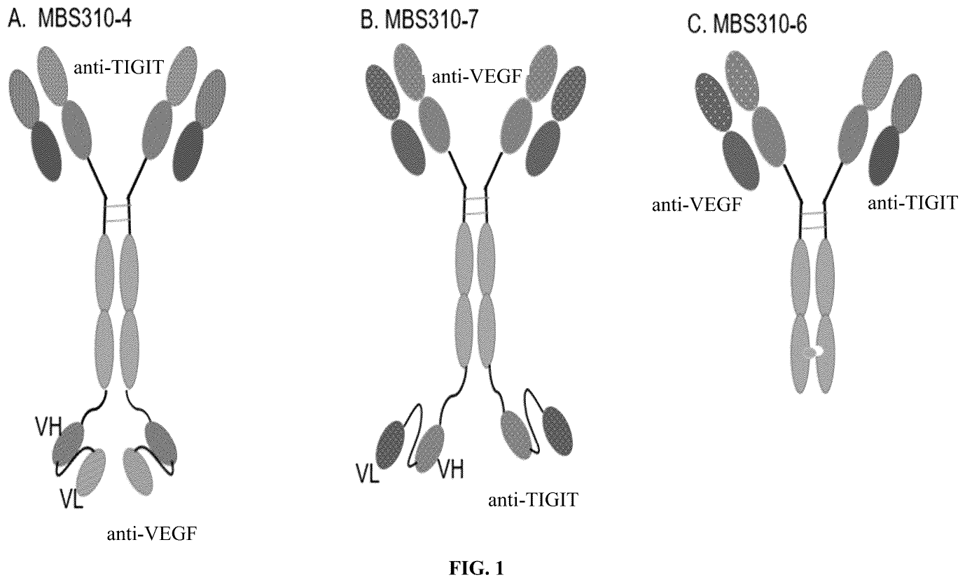

Bispecific antibodies were constructed in either a symmetrical format or an asymmetrical format, with the structures shown in FIG. 1. The symmetrical bispecific antibodies included MBS310-4 and MBS310-7, which both contained two TIGIT binding domains and two VEGF binding domains, while the asymmetrical bispecific antibodies included MBS310-6 which contained one TIGIT binding domain and one VEGF binding domain. The TIGIT binding domain used the heavy and light chain variable regions comprising the amino acid sequences of SEQ ID NOs: 13 and 14, respectively, and the VEGF binding domain used Avastin® bevacizumab's heavy chain and light chain variable region sequences, i.e., SEQ ID NOs: 15 and 16.

In particular, MBS310-4 contained a long chain of SEQ ID NO: 26 (anti-TIGIT heavy chain variable region-heavy chain constant region-linker-anti-VEGF heavy chain variable region-linker-anti-VEGF light chain variable region) and a short chain of SEQ ID NO: 22 (anti-TIGIT light chain variable region-light chain constant region); MBS310-7 contained a long chain of SEQ ID NO: 25 (anti-VEGF heavy chain variable region-heavy chain constant region-linker-anti-TIGIT heavy chain variable region-linker-anti-TIGIT light chain variable region) and a short chain of SEQ ID NO: 24 (anti-VEGF light chain variable region-light chain constant region); and MBS310-6 contained an anti-VEGF heavy chain variable region-heavy chain constant region (with knob) chain of SEQ ID NO: 23, an anti-TIGIT heavy chain variable region-heavy chain constant region (with hole) chain of SEQ ID NO: 21, an anti-VEGF light chain variable region-light chain constant region chain of SEQ ID NO: 24, and an anti-TIGIT light chain variable region-light chain constant region chain of SEQ ID NO: 22.

DNA fragments encoding the chains above were synthesized. Those coding for the short (light) chains were digested with ClaI and HindIII, those coding for the long (heavy) chains were digested with EcoRI and XhoI, the pCMV-plasmids were digested with HindIII and EcoRI, and the GS-vectors were digested with ClaI and XhoI. The DNA fragments were recovered, ligated, and transformed into bacteria. Single bacterial colonies were picked up and sequenced, and expression vectors containing the correct sequences were obtained. MBS310-4 and MBS310-7 used the single-cell expression system, while MBS310-6 employed the dual-cell expression system.

HEK-293F cells (Cobioer, China) were transfected with the expression vectors obtained above using PEI. Briefly, the HEK-293F cells were transfected with the expression vectors using polyethyleneinimine (PEI) at a DNA:PEI ratio of 1:3, 1.5 gg of DNAs per millimeter of cell medium. Transfected HEK-293F cells were cultured in an incubator at 37° C. under 5% CO₂ with shaking at 120 RPM. After 10-12 days, the cell culture supernatants were harvested, centrifuged at 3500 rpm, and flowed through a 0.22 μm film filter to remove the cell debris. The proteins as expressed were purified using pre-equilibrated Protein-A affinity columns (Cat #:17040501, GE, USA) and eluted with the elution buffer (20 mM citric acid, pH 3.0-3.5). The obtained antibodies, including the half antibodies, were kept in PBS buffer (pH 7.0) and the concentrations were determined using a NanoDrop analyzer.

### Example 3. Assembly of Exemplary Asymmetrical Bispecific Antibodies

The purified half-antibodies were assembled in vitro to generate the MBS310-6 molecules. Briefly, the two half antibodies, MBS310-6-knob and MBS310-6-hole, were mixed at 1:1 molar ratio. The mixtures were added with Tris base buffer till pH 8.0 followed by reducing agent glutathione (GSH), and allowed to react overnight at 25° C. with low-speed stirring. Then, the mixtures were added with 2 M acetic acid solution to adjust pH to 5.5. The reducing agent was removed by ultrafiltration, to terminate the reaction.

The antibodies were purified using anions exchange chromatography followed by cation exchange chromatography. Anion exchange columns were balanced with low-salt Tris buffer (pH8.0), and loaded with the antibody samples. The components that had passed through the columns were collected, and rinsed by low-salt Tris buffer (pH8.0) until UV280 trended to the baseline. The collected samples were adjusted to pH5.5 using an acetic acid solution, concentrated to 1 ml using a 30 kDa ultrafilter tube, and filtered using 0.2 μm membrane. Then, cation exchange columns were balanced with a low-concentration acetate buffer (pH5.5), and loaded with the antibody samples. The low-concentration acetate buffer (pH5.5) was used to balance the columns again, and elution was done using 20 CV acetate solutions (concentration at 0-100%, pH5.5). The purified antibodies with a purity higher than 90% as measured by mass spectrum, were further characterized below.

### Example 4. Binding Capability of Exemplary Anti-VEGF/TIGIT Bispecific Antibodies to VEGF

The purified bispecific antibodies were tested for their binding capability to recombinant human/monkey and mouse VEGF molecules by ELISA, wherein the human and monkey VEGF molecules had the same sequence.

Briefly, an ELISA plate was coated with 100 μl 500 ng/ml human VEGF-A molecules (Cat #:11066-HNAN, Sino Biological, CN), mouse VEGF-A molecules (Cat #:50159-MNAB, Sino Biological, CN), human VEGF-B-his molecules (Cat #:VE6-H5225, Acrobiosystems Co., CN) and human VEGF-C-his molecules (Cat #:VEC-H4225, Acrobiosystems Co., CN) respectively overnight at 4° C. The plate was blocked with 200 μl blocking buffer (PBS+1% BSA+1% goat serum+0.05% Tween 20) at room temperature for 2 h, added with 100 μl serially diluted anti-TIGIT/VEGF bispecific molecules of the disclosure or bevacizumab (as the positive control, heavy chain with GenBank accession no.: AOZ48530.1 (Front Plant Sci 7, 1156 (2016)), light chain with GenBank accession no.: 2FJH_L (J. Biol. Chem. 281 (10), 6625-6631 (2006)), with the highest concentration at 40 μg/ml, and incubated at room temperature for 1 h. The ELISA plate was washed with PBST (PBS+0.05% Tween 20) for three times, added with HRP-goat anti-mouse IgG (1:5000, Cat #:A9309-1 ml, Sigma, USA), and incubated at room temperature for 1 h. The ELISA plate was added with freshly prepared Ultra-TMB (Cat #:555214, BD, USA), and left still for 5 min for color development. The absorbance was read at 450 nm using SpectraMax® i3X microplate reader.

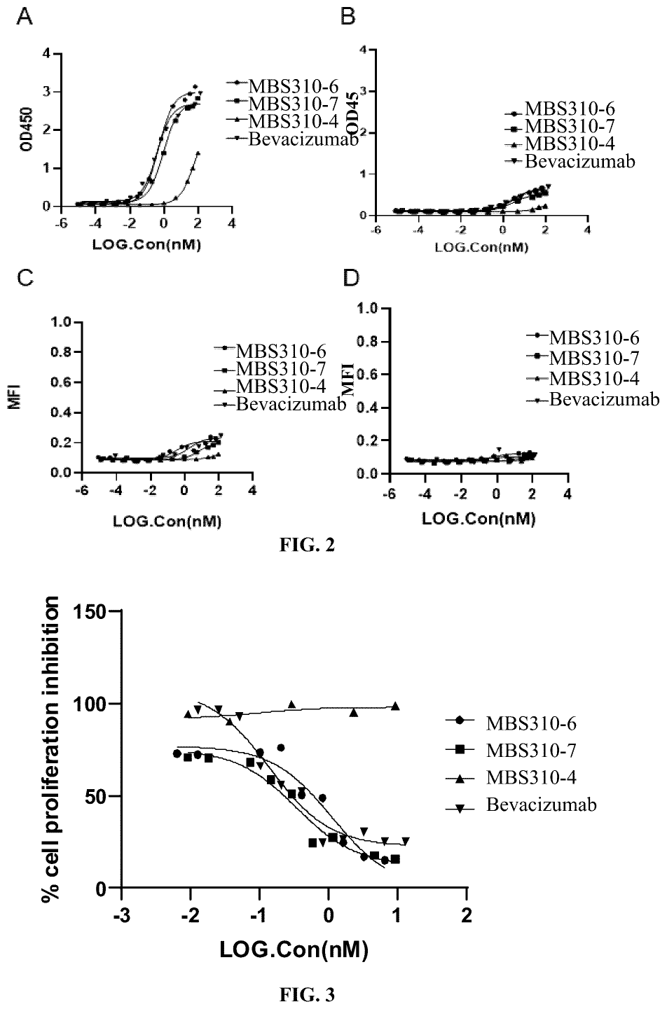

The results were shown in FIG. 2, MBS310-6 and MBS30-7 had high binding capability to human and monkey VEGF-A (A), weak binding capability to mouse VEGF-A (B) and no binding to human VEGF-B (C) and VEGF-C (D), which was comparable to that of bevacizumab, while MBS310-4, probably due to its structure, showed much lower binding capability to VEGF-A (A).

### Example 5. Inhibitory Effect of Exemplary Anti-TIGIT/VEGF Bispecific Antibodies on HUVEC Cell Proliferation

The VEGF molecules can promote proliferation of vascular endothelial cells. The bispecific antibodies of the disclosure were tested for their inhibitory effect on human umbilical vein endothelial cell (HUVEC) proliferation according to the method described in Gospodarowicz D et al., (1989) PNAS, 86:7311).

Briefly, a 96-well cell culture plate was added with 0.2 ml culture medium containing 1×10⁴ HUVECs (Cat #: CC-2517, Lonza, USA), VEGF molecules (Cat #: 11066-HNAN, Sino Biological, CN) at the final concentration of 25 ng/ml and serially diluted bispecific antibodies (2-fold dilution starting at 20 μg/ml final concentration). The plate was kept in an incubator at 37° C. with 5% CO₂ for 72 h. The cells were counted using the CCK8 test kit (Cat #: CK04, Dojindo, JP). Specifically, the plate was added with 20 μl of the CCK9 solution, incubated at 37° C. for 2 h, and determined for the absorbance at 450 nm.

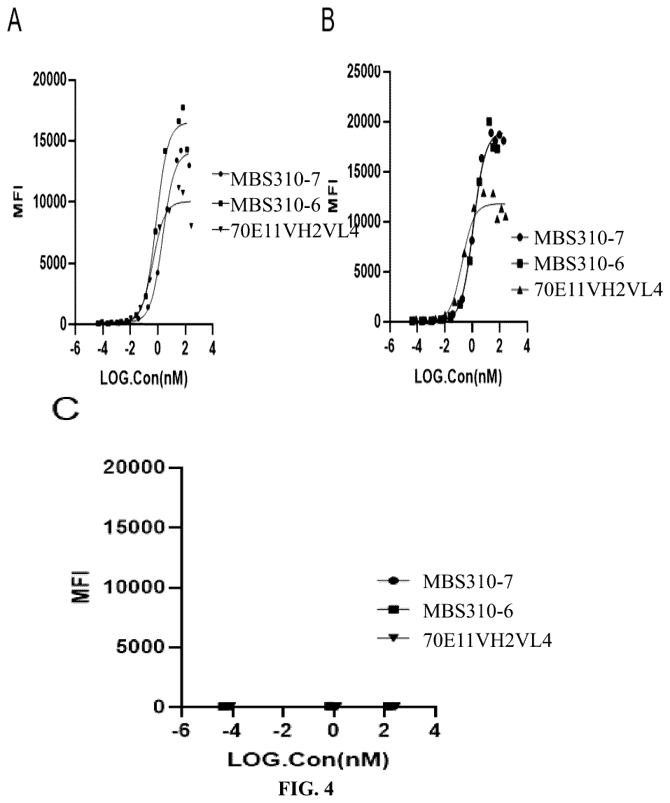

The results were shown in FIG. 3. In particular, similar to bevacizumab, MBS310-6 and MBS310-7 significantly inhibited VEGF-mediated HUVEC proliferation. However, due to its decreased binding capability to VEGF, MBS310-4 showed no effect on HUVEC proliferation.

### Example 6. Binding Capability of Exemplary Anti-TIGIT/VEGF Bispecific Antibodies to Cell Surface Human/Monkey TIGIT Molecules

The bispecific molecules were further tested for their binding capability to cell surface human, monkey and mouse TIGIT molecules by FACS, using the HEK293A cell lines generated in Example 1.

Briefly, 10⁵ HEK293A cells in 100 μl cell culture medium were seeded onto a 96-well plate, which was later added with 50 μl serially diluted bispecific antibodies of the disclosure. After incubation at 4° C. for 1 h, the plate was washed with PBS for three times, added with APC-goat-anti-mouse IgG (1:500, Cat #: 405308, BioLegend, USA). After incubation at 4° C. for 1 h, the plate was washed with PBS for three times, and measured for fluorescence using a cytometry (BD). An anti-TIGIT antibody 70E11VH2VL4 was used as the control, which contained the heavy and light chain variable regions of SEQ ID NOs: 13 and 14 (same with the anti-TIGIT heavy and light chain variable regions in the bispecific molecules of the disclosure) and the heavy and light chain constant regions of SEQ ID NOs: 19 (X1=T, X2=L, X3=Y) and 20.

The results were shown in FIG. 4. MBS310-6 and MBS310-7, similar to 70E11VH2VL4, had high binding capability to human and monkey TIGIT (A, B), but did not bind mouse TIGIT (C), indicating the structure of the bispecific antibodies as represented by MBS310-6 and MBS310-7 had no adverse effect on TIGIT binding capability.

### Example 7. Effect of Free VEGF Molecules on Binding of Exemplary Anti-TIGIT/VEGF Bispecific Antibodies to Cell Surface Human/Monkey TIGIT Molecules

The bispecific antibodies of the disclosure were tested for their binding capability to TIGIT⁺ cells in the presence of free VEGF molecules, using the HEK293A cell lines generated in Example 1.

Briefly, 10⁵ HEK293A cells in 100 μl cell culture medium were seeded onto a 96-well plate, which was later added with 50 μl serially diluted bispecific antibodies of the disclosure and 50 μl free human VEGF-A molecules at the final concentration of 0 ng/ml, 50 ng/ml or 50 μg/ml (Cat #:11066-HNAN, Sino Biological, CN). After incubation at 4° C. for 1 h, the plate was washed with PBS for three times, and added with APC-goat-anti-mouse IgG (1:500, Cat #: 405308, BioLegend, USA). After incubation at 4° C. for 1 h, the plate was washed with PBS for three times, and measured for fluorescence using a cytometry (BD).

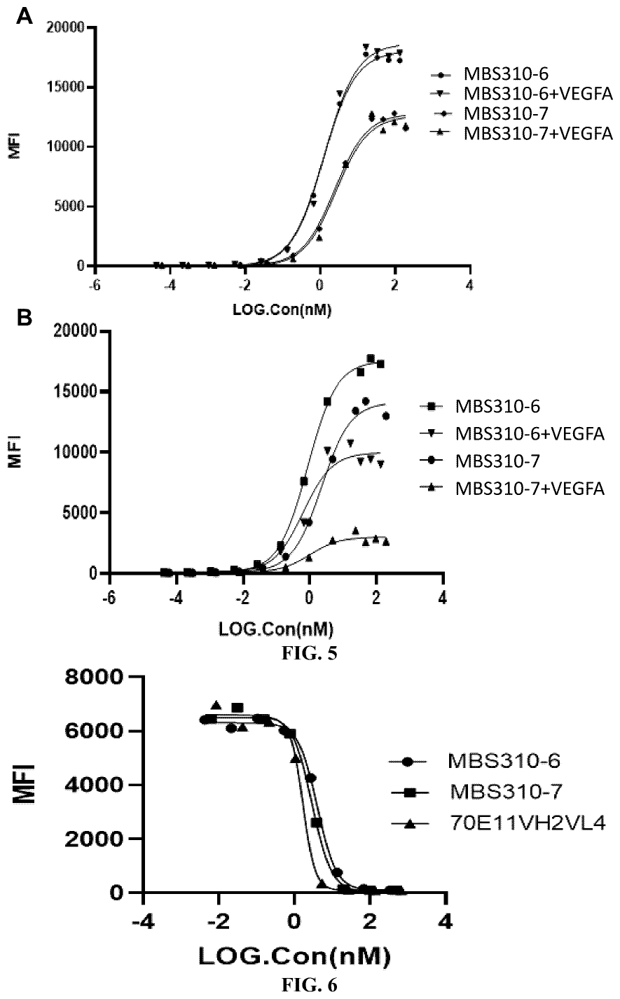

The results were shown in FIG. 5. The presence of 50 ng/ml VEGF-A molecules did not affect the binding of MBS310-6 and MBS310-7 with human TIGIT-expressing HEK293A cells (A), but the presence of 50 μg/ml VEGF-A molecules did (B), especially to MBS310-7.

### Example 8. Inhibitory Effect of Exemplary Anti-TIGIT/VEGF Bispecific Antibodies on TIGIT-PVR Interaction

Studies have indicated PVR is the main ligand for TIGIT. The inhibitory effect of the exemplary anti-TIGIT/VEGF bispecific antibodies on TIGIT-PVR interaction was assayed by FACS using the A549/human PVR cells generated in Example 1.

Briefly, serially diluted bispecific antibodies were mixed and incubated with TIGIT-mFc molecules (Cat #: 10917-H38H, Sino Biological, CN) at the final concentration of 5 μg/ml at 37° C. for 1 h. A 96-well plate was seeded with 10⁵ A549/human PVR cells in 100 μl cell culture medium, and then added with 100 μl the antibody/TIGIT-mFc mixture. After incubation at 4° C. for 1 h, the plate was washed with PBS for three times, and then added with PE-goat-anti-mouse IgG (1:500, Cat #:31861, Thermofisher, USA). After incubation at 4° C. for 1 h, the plate was washed with PBS for three times, and measured for fluorescence using a cytometry (BD).

According to FIG. 6, MBS310-6 and MBS310-7, similar to the monospecific anti-TIGIT antibody 70E11VH2VL4, significantly blocked TIGIT-PVR binding or interaction.

### Example 9. Effect of Exemplary Anti-TIGIT/VEGF Bispecific Antibodies on T Cell Activation

The effect of the anti-VEGF/TIGIT bispecific antibodies on T cell activity was tested by T cell viability assay.

Briefly, PBMCs from healthy human donors' blood samples were collected by density gradient centrifugation, and suspended in RPMI1640 medium. CD4⁺ T cells were isolated from the PBMCs using Invitrogen Dynabeads™ Untouched™ human CD4⁺ T cells kit (Cat #:11346D, Thermal Fisher Scientific, USA). The CD4⁺ T cells were suspended in RPMI complete medium (90% RPMI medium+10% FBS) at the cell density of 1.0×10⁶/ml, added with Dynabeads™ human T-activator CD3/CD28 (Cat #: 11132D, Gibco, USA), and cultured for 10 days at 37° C. with 5% CO₂.

The CD4⁺ T cells were harvested, washed with RPIM medium for three times, and adjusted to the cell density of 2×10⁵/ml. A 96-well plate was coated with 50 μl 0.25 μμg/ml anti-CD3 antibody (OKT3, Cat #:GMP-10977-H001, Sino Biological, CN) and 50 μl recombinant PVR-hFc proteins (Cat #:10109-H02H, Sino Biological, CN) at 4° C. overnight. The plate was washed with PBS for three times, and then blocked with PBS buffer containing 1% bovine serum albumin at 37° C. for 90 min. The plate was washed with PBS for three times, and added with 150 μl CD4⁺ T cell suspensions and 50 μl serially diluted bispecific antibodies of the disclosure. The cells were cultured at a 37° C. incubator for 3 days. 70E11VH2VL4 and Tiragolumab were used as controls. The cell culture supernatants were collected for determination of IFN-γ and IL-2 levels using human IFN-gamma ELISA kit (Cat #: SIF50, R&D, USA) and human IL-2 Quantikine® ELISA kit (Cat #: S2050, R&D, USA). The assay was done in triplicate.

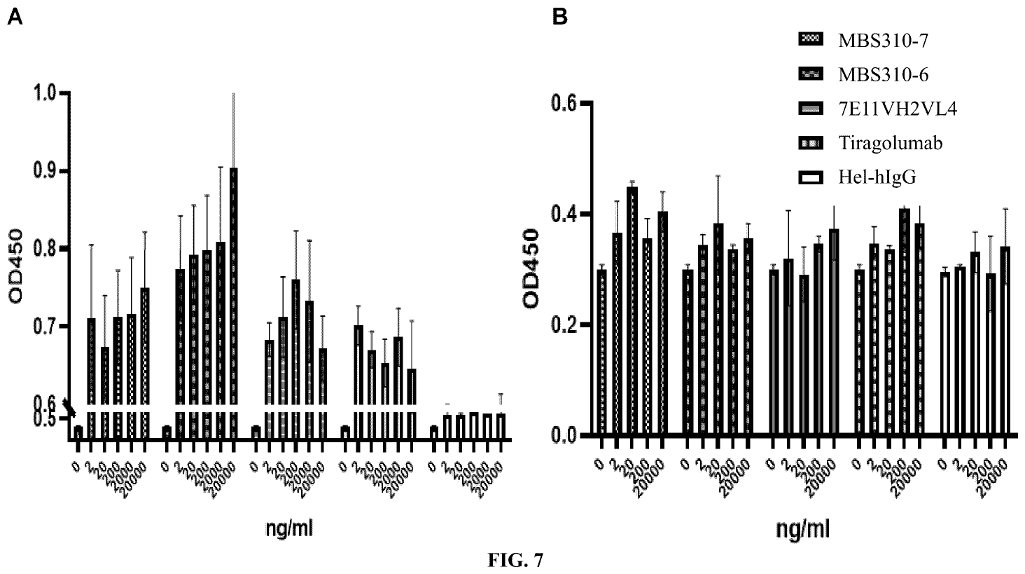

According to FIG. 7, all antibodies, including the monospecific anti-TIGIT antibody (70E11VH2VL4 and Tiragolumab), and the bispecific antibodies of the disclosure, improved T cell activity and increased IFN-γ secretion by T cells (FIG. 7 (A)), wherein MBS310-6 showed the highest activity in T cell activation. Particularly, as compared to the anti-Hel antibody, these antibodies increased IFN-γ secretion by T cells in a concentration dependent manner Further, 70E11VH2VL4, MBS310-6 and MBS310-7 showed higher activity in T cell activation than Tiragolumab at certain concentrations.

### Example 10. Exemplary Anti-TIGIT/VEGF Bispecific Antibodies Induced ADCC Against TIGIT⁺ Cells

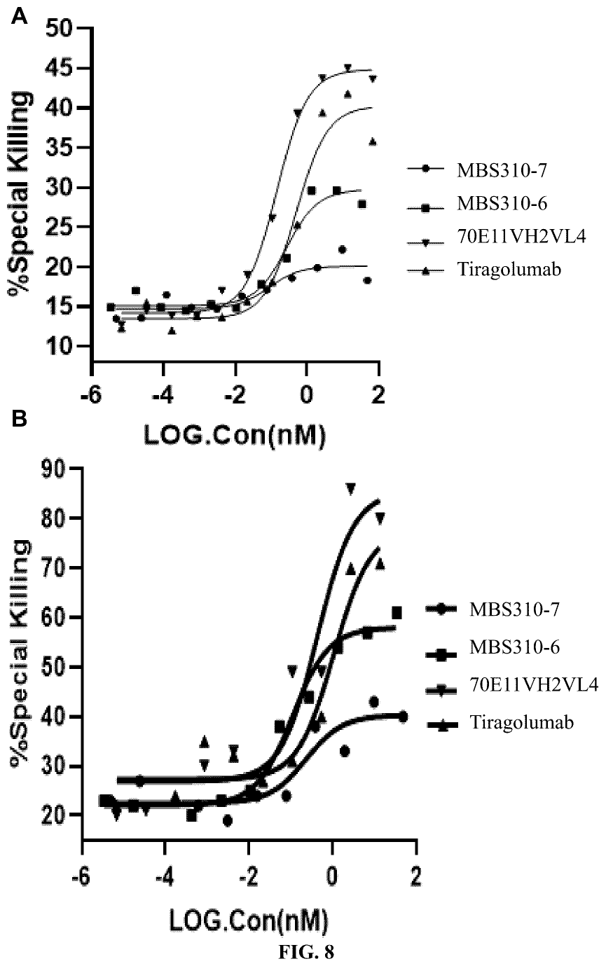

The bispecific antibodies of the disclosure were tested for their ability to induce NK92 cell-mediated ADCC against TIGIT⁺ cells using the HEK293A/human TIGIT cells as generated in Example 1. Briefly, the HEK293A/human TIGIT cells and NK92MI-CD16a (as the effector cells, Huabo Bio) were centrifuged at 1200 rpm for 5 min, and then suspended in the ADCC assay culture medium (MEM medium (Cat #:12561-056, Gibco)+1% FBS (Cat #:FND500, EX-cell)+1% BSA (Cat #:V900933-1KG, VETEC)), wherein the cell viability was about 90%. Then, 50 μl HEK293A/human TIGIT cells at the cell density of 4×10⁵/ml, and 50 μl NK92MI-CD16a cells at the cell density of 2×10⁶/ml were added to a 96-well plate, with the effector-target ratio at 5:1. The plate was respectively added with antibodies, including the bispecific antibodies of the disclosure, at the final concentration of 50000 ng/ml, 10000 ng/ml, 2000 ng/ml, 400 ng/ml, 80 ng/ml, 16 ng/ml, 3.2 ng/ml, 0.64 ng/ml, 0.128 ng/ml, and 0.0256 ng/ml, incubated at 37° C. for 4 h, and added with LDH developing solutions (Cytotoxicity Detection Kit PLUS (LDH), Cat #:04744926001, Roche), 100 μl per well. The plate was kept in dark at room temperature for 20 min and read in a MD SpectraMax i3. Tiragolumab was used as a positive control. The results were shown in FIG. 8 (A) and Table 1.

The bispecific antibodies of the disclosure were further tested for their ability to induce PBMC-mediated ADCC against TIGIT⁺ cells using the HEK293A/human TIGIT cells as generated in Example 1, wherein the pLV-EGFP(2A)-Puro plasmids transfected into the HEK293 cells express green fluorescent proteins (GFPs). Briefly, PBMCs from healthy human donors' blood samples were collected by density gradient centrifugation, and cultured in cell culture medium (RIPM1640+10% FBS+300IU IL-2) overnight. The target cells and PBMCs (as the effector cells) were centrifuged at 1200 rpm for 5 min, and then suspended in the ADCC assay culture medium (MEM medium+1% FBS), wherein the cell viability was about 90%. Then, 50 μl HEK293A/human TIGIT cells at the cell density of 4×10⁵/ml, and 50 μl PBMCs at the cell density of 8×10⁶/ml were added to a 96-well plate, with an effector-target ratio at 20:1. The plate was added respectively with antibodies, including the bispecific antibodies of the disclosure, at the final concentration of 50000 ng/ml, 10000 ng/ml, 2000 ng/ml, 400 ng/ml, 80 ng/ml, 16 ng/ml, 3.2 ng/ml, 0.64 ng/ml, 0.128 ng/ml, and 0.0256 ng/ml, incubated at 37° C. for 24 h, washed with PBS for three times, and then incubated with the stain from Fixable violet dead cell stain kit (Cat #: L34964, Thermo Fisher, USA) at 37° C. for 30 min. The cells were washed with PBS for three times, and subjected to FACS. The death rate of GFP⁺ cells, i.e., the HEK293A/human TIGIT cells, was determined, and the assay results were shown in FIG. 8 (B) and Table 1.

As shown in FIG. 8 and Table 1, all antibodies induced NK92MI-CD16a or PBMC-mediated TIGIT⁺ cell death, with MBS310-6 and MBS310-7 induced higher ADCC than the monospecific antibodies. Particularly, MBS310-6 induced higher ADCC than MBS310-7.

TABLE 1
Ability of bispecific antibodies to induce ADCC

| EC₅₀ | MBS310-7 | | MBS310-6 | 70E11VH2VL4 | Tiragolumab |
|---|---|---|---|---|---|
| NK92 | 0.08456 | nM | 0.2675 nM | 0.1416 nM | 0.5113 nM |
| PBMC | 0.2393 | nM | 0.1829 nM | 0.1616 nM | 0.3107 nM |

### Example 11. Binding Affinity of Exemplary Anti-TIGIT/VEGF Bispecific Antibodies to Human TIGIT and VEGF

Using BIAcore™ 8K instrument (GE Life Sciences, US), the binding affinity of the bispecific molecules of the disclosure to human TIGIT and VEGF was quantitatively measured. Briefly, 100-200 response units (RU) of human TIGIT-his protein (Cat #:10917-H08H, Sino Biological, CN) or human VEGF-A (Cat #:11066-HNAN, Sino Biological, CN) were coupled to CMS biosensor chips (Cat #:BR-1005-30, GE Life Sciences, US). The un-reacted groups were then blocked with 1M ethanolamine Serially diluted antibodies at concentrations ranging from 0.3 μM to 10 μM were injected into the SPR running buffer (HBS-EP buffer, pH7.4, Cat #:BR-1006-69, GE Life Sciences, US) at 30 μL/min. The binding affinity was calculated with the RUs of blank controls subtracted, and the association rate (kₐ) and dissociation rate (k_{d}) were determined using the one-to-one Langmuir binding model (BIA Evaluation Software, GE Life Sciences, US). The equilibrium dissociation constant K_{D} was calculated as the k_{d}/kₐ ratio.

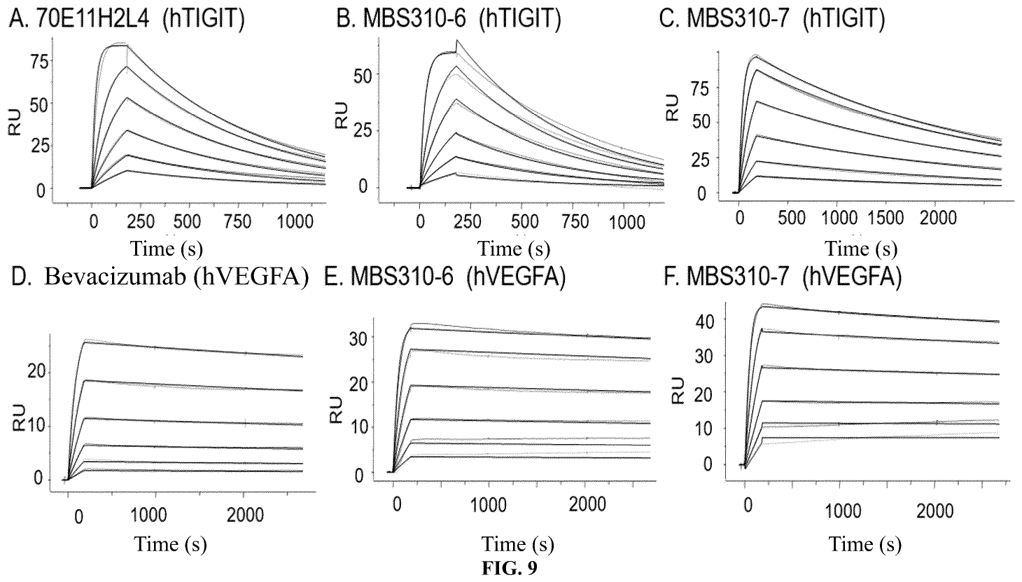

According to the SPR binding curves (FIG. 9 (A-F)), the binding affinity of the bispecific molecules of the disclosure to human TIGIT and VEGF was determined and summarized in Table 2.

TABLE 2
Binding affinity of bispecific antibodies to human TIGIT and VEGF

| | human TIGIT | | | human VEGF-A | | |
|---|---|---|---|---|---|---|
| Ab ID | Ka | Kd | K_{D} | Ka | Kd | K_{D} |
| 70E11VH2VL4 | 1.85E+05 | 1.49E−03 | 8.05E−09 | / | / | / |
| bevacizumab | / | / | / | 2.06E+05 | 4.43E−05 | 2.15E−10 |
| MBS310-6 | 1.44E+05 | 1.84E−03 | 1.28E−08 | 1.89E+05 | 3.14E−05 | 1.66E−10 |
| MBS310-7 | 1.01E+05 | 4.20E−04 | 4.15E−9 | 3.47E+05 | 4.39E−05 | 1.27E−10 |

The bispecific antibodies of the disclosure were further tested by SPR for their capability to bind two antigens simultaneously. For the assay measuring VEGF binding affinity followed by TIGIT binding affinity, MBS310-6 and MBS310-7 were respectively coupled to a CMS biosensor chip (anti-human Fc, Cat #: 10266084, GE Life Sciences, USA) at 1 μg/ml. Serially diluted VEGF molecules (2-fold dilution starting at 2 μg/ml) and serially diluted TIGIT molecules (2-fold dilution starting at 4 μg/ml) were injected into the SPR running buffer in said order at 30 μL/min. For the assay measuring TIGIT binding affinity followed by VEGF binding affinity, MBS310-6 and MBS310-7 were respectively coupled to a CMS biosensor chip (Cat #: 10266084, GE Life Sciences, USA) at 4 μg/ml. Serially diluted TIGIT molecules (2-fold dilution starting at 4 μg/ml) and serially diluted VEGF molecules (2-fold dilution starting at 2 μg/ml) were injected into the SPR running buffer in said order at 30 μL/min. The first antigen-antibody association kinetics was followed for 180 s and the dissociation kinetics was followed for 500 s. Then, the second antigen-antibody association kinetics was followed for 180 s and the dissociation kinetics was followed for no less than 500 s. The binding affinity was calculated with the RUs of blank controls subtracted.

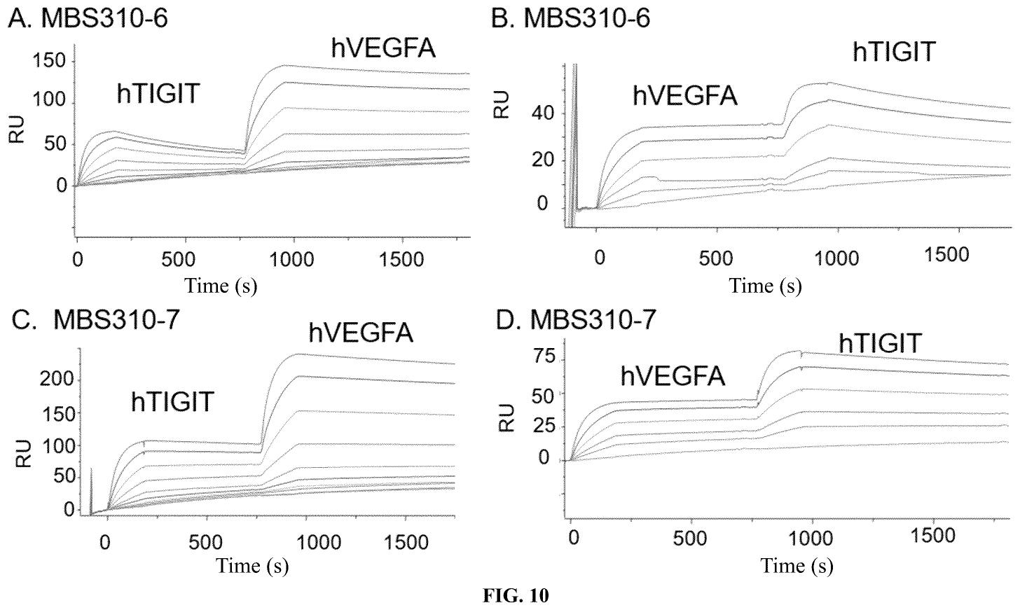

The results were shown in FIG. 10. MBS310-6 and MBS310-7 were able to bind VEGF and TIGIT simultaneously independent of the antigen exposure order, and the kinetics data was quite consistent to those obtained when the binding affinity to single antigens was measured.

### Example 12. Exemplary Afucosylated Anti-TIGIT/VEGF Bispecific Antibodies Induced ADCC Against TIGIT⁺ Cells

The nucleotides encoding MBS310-6 were inserted into pCDNA3.1 (Invitrogen, Carlsbad, USA) to generate expression vectors which were later transfected into CHO-K1-AF cells with Slc35C1 knockdown (in house prepared, see US2018/0022820 A1). Half antibodies were expressed, purified, and assembled according to the protocol in Example 2 and Example 3, to generate afucosylated MBS310-6 antibodies, referred to as MBS310-6-AF herein.

The bispecific antibody MBS310-6-AF was tested for its ability to induce NK92 cell-mediated ADCC against TIGIT⁺ cells, using the NK92MI-CD16a cells as the effector cells and the HEK293A/human TIGIT cells generated in Example 1 as the target cells, following the protocol of Example 10 with minor modification as described below.

Briefly, the HEK293A/human TIGIT cells and NK92MI-CD16a (Huabo Bio) were centrifuged at 1200 rpm for 5 min, and then suspended in the ADCC assay culture medium (MEM medium (Cat #:12561-056, Gibco)+1% FBS (Cat #:FND500, EX-cell)+1% BSA (Cat #:V900933-1KG, VETEC)), wherein the cell viability was about 90%. Then, 50 μl HEK293A/human TIGIT cells at the cell density of 4×10⁵/ml, and 50 μl NK92MI-CD16a cells at the cell density of 2×10⁶/ml were added to a 96-well plate, with the effector-target ratio at 5:1. The plate was respectively added with antibodies, including the bispecific antibodies of the disclosure, at the final concentration of 50000 ng/ml, 10000 ng/ml, 2000 ng/ml, 400 ng/ml, 80 ng/ml, 16 ng/ml, 3.2 ng/ml, 0.64 ng/ml, 0.128 ng/ml, and 0.0256 ng/ml, incubated at 37° C. for 4 h, washed with PBS for three times, and then incubated with the stain from Fixable violet dead cell stain kit (Cat #: L34964, Thermo Fisher, USA) at 37° C. for 30 min. The cells were washed with PBS for three times, added with 2 μl PE-mouse anti-human CD69 antibody (Cat #:555531, BD, USA), incubated at 37° C. for 30 min, centrifuged, washed with PBS for three times, and then subjected to FACS. The death rate of GFP⁺ cells, i.e., the HEK293A/human TIGIT cells was calculated, and the mean fluorescence intensity was determined for the GFP⁻ cells, i.e., the NK92MI-CD16a cells.

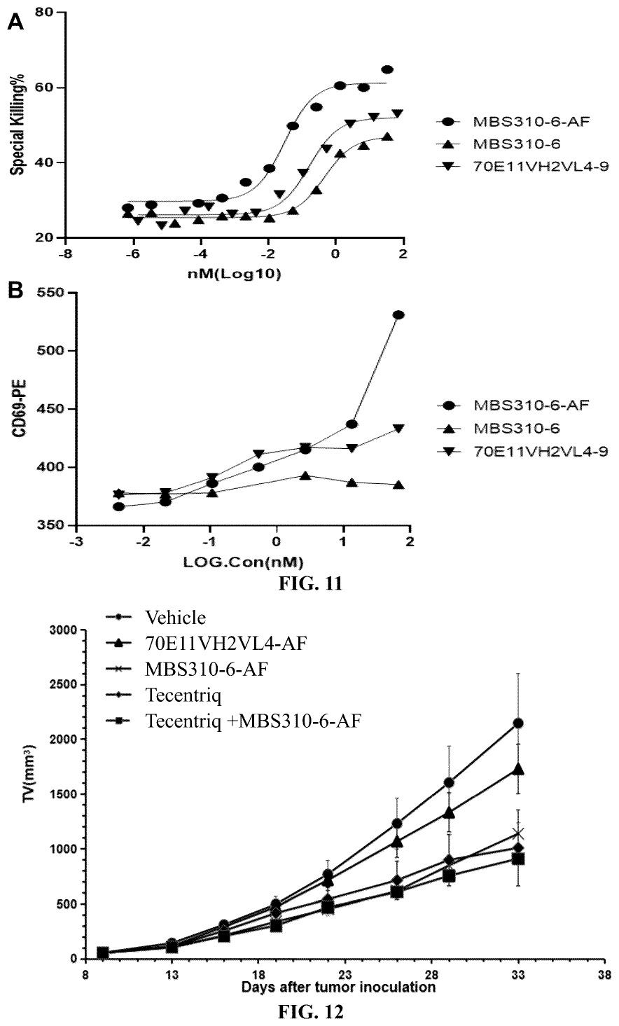

As shown in FIG. 11 (A), the afucosylation significantly increased the ADCC induced by MBS310-6, i.e., MBS310-6-AF caused more target cell death than MBS310-6 and even 70E11VH2VL4 from which its TIGIT binding domains were derived. In the meanwhile, MBS310-6-AF evidently enhanced NK cell activation, as the CD69 expression level on NK92 cells was significantly higher than that induced by MBS310-6 or 70E11VH2VL4, as shown in FIG. 11 (B).

### Example 13. In Vivo Anti-Tumor Effect of Exemplary Anti-TIGIT/VEGF Bispecific Antibodies

The in vivo anti-tumor activity of MBS310-6-AF, 70E11VH2VL4-AF and Tecentriq® Atezolizumab (an anti-PD-L1 antibody) was tested, wherein all these antibodies contained human IgG1 and κ constant regions, and MBS310-6-AF and 70E11VH2VL4-AF's Fc regions were afucosylated.

Briefly, the mice implanted with human non-small-cell lung cancer cells (NSCLC030) were sacrificed when the tumor sizes reached 500 to 800 mm³. The tumors were collected from the mice, cut into pieces of 2 mm×2 mm×2 mm, and injected subcutaneously into 6-8-week-old male NCG mice (GemPharmatech, NJ, CN) at the right flank using trocars on Day 0, one piece per mouse. The mice were then injected with 2×10⁶ PBMCs from healthy donors. On day 9 when the tumor sizes were around 50-70 mm³, the animals were allocated into five groups according to the tumor sizes, eight mice per group. The mice were intraperitoneally administered with MBS310-6-AF (20 mg/kg), 70E11VH2VL4-AF (10 mg/kg), Atezolizumab (5 mg/kg), MBS310-6-AF (20 mg/kg)+Atezolizumab (5 mg/kg), and PBS, respectively, on Day 9, 12, 16, 19, 23, 26 and 30.

Tumor sizes and mouse weights were monitored over time. In specific, the tumor size was determined by measuring by a caliper the length (the longest diameter) and the width (the diameter perpendicular to the length) of a tumor and calculating the volume as 0.5×D×d². The test was terminated before the tumor sizes in the administration group reached 3.5 cm³. One-way ANOVA was used to identify tumor size differences among groups.

The results were shown in FIG. 12. It can be seen that MBS310-6-AF significantly inhibited tumor growth, and its anti-tumor efficacy was much better than the monospecific antibody 70E11VH2VL4-AF. Atezolizumab also showed potent inhibitory effect on tumor growth, and the combination of MBS310-6-AF and Atezolizumab provided even higher anti-tumor efficacy.

Exemplary sequences in the present application are summarized below.

Description/Sequence and SEQ ID NO.

VH-CDR1 of 70E11VH2VL4 and TIGIT binding
domain in MBS310-4, MBS310-6 and MBS310-7
SYNVH
(SEQ ID NO: 1)
VH-CDR2 of 70E11VH2VL4 and TIGIT binding
domain in MBS310-4, MBS310-6 and MBS310-7
TIYPGNLATSYNQKFKG
(SEQ ID NO: 2)
VH-CDR3 of TIGIT binding
domain in 70E11VH2VL4, MBS310-4, MBS310-6
and MBS310-7
SGTMDY
(SEQ ID NO: 3)
VL-CDR1 of 70E11VH2VL4 and TIGIT binding
domain in MBS310-4, MBS310-6 and MBS310-7
RASSSISSTYLH
(SEQ ID NO: 4)
VL-CDR2 of 70E11VH2VL4 and TIGIT binding
domain in MBS310-4, MBS310-6 and MBS310-7
NTQNLAS
(SEQ ID NO: 5)
VL-CDR3 of 70E11VH2VL4 and TIGIT binding
domain in MBS310-4, MBS310-6 and MBS310-7
QQFGGYPLIT
(SEQ ID NO: 6)
VH-CDR1 of bevacizumab and VEGF binding
domain in MBS310-4, MBS310-6 and MBS310-7
NYGMN
(SEQ ID NO: 7)
VH-CDR2 of bevacizumab and VEGF binding
domain in MBS310-4, MBS310-6 and MBS310-7
WINTYTGEPTYAADFKR
(SEQ ID NO: 8)
VH-CDR3 of bevacizumab and VEGF binding
domain in MBS310-4, MBS310-6 and MBS310-7
YPHYYGSSHWYFDV
(SEQ ID NO: 9)
VL-CDR1 of bevacizumab and VEGF binding
domain in MBS310-4, MBS310-6 and MBS310-7
SASQDISNYLN
(SEQ ID NO: 10)
VL-CDR2 of bevacizumab and VEGF binding
domain in MBS310-4, MBS310-6 and MBS310-7
FTSSLHS
(SEQ ID NO: 11)
VL-CDR3 of bevacizumab and VEGF binding
domain in MBS310-4, MBS310-6 and MBS310-7
QQYSTVPWT
(SEQ ID NO: 12)
VH of 70E11VH2VL4
QVQLVQSGAEVKKPGASVKVSCKASGYTFTSYNVH
WVRQAPGQGLEWMGTIYPGNLATSYNQKFKGRVTL
TADTSTSTVYMELSSLRSEDTAVYYCARSGTMDYW
GQGTTVTVSS
(SEQ ID NO: 13)
VL of 70E11VH2VL4
EIVLTQSPGTLSLSPGERATMTCRASSSISSTYLH
WYQQKPGASPKLLIYNTQNLASGVPARFSGSGSGT
SYTLTISRLEPEDFAVYYCQQFGGYPLITFGAGTK
LELK
(SEQ ID NO: 14)
VH of bevacizumab
EVQLVESGGGLVQPGGSLRLSCAASGYTFTNYGMN
WVRQAPGKGLEWVGWINTYTGEPTYAADFKRRFTF
SLDTSKSTAYLQMNSLRAEDTAVYYCAKYPHYYGS
SHWYFDVWGQGTLVTVSS
(SEQ ID NO: 15)
VL of bevacizumab
DIQMTQSPSSLSASVGDRVTITCSASQDISNYLNW
YQQKPGKAPKVLIYFTSSLHSGVPSRFSGSGSGTD
FTLTISSLQPEDFATYYCQQYSTVPWTFGQGTKVE
IKR
(SEQ ID NO: 16)
Linker
GGGGSGGGGSGGGGS
(SEQ ID NO: 17)
GGGGSGGGGSGGGGSGGGGS
(SEQ ID NO: 18)
Heavy chain constant region
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSREEMTKNQ
VSLX1CX2VKGFYPSDIAVEWESNGQPENNYKTTP
PVLDSDGSFFLX3SKLTVDKSRWQQGNVFSCSVMH
EALHNHYTQKSLSLSPGK
(SEQ ID NO: 19)
Wildtype heavy chain constant region
SEQ ID NO: 19, X1 = T, X2 = L, X3 = Y
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSREEMTKNQ
VSLTCLVKGFYPSDIAVEWESNGQPENNYKTTPPV
LDSDGSFFLYSKLTVDKSRWQQGNVFSCSVMHEAL
HNHYTQKSLSLSPGK
heavy chain constant region with
knob mutations
SEQ ID NO: 19, X1 = W, X2 = L, X3 = Y
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSREEMTKNQ
VSLWCLVKGFYPSDIAVEWESNGQPENNYKTTPPV
LDSDGSFFLYSKLTVDKSRWQQGNVFSCSVMHEAL
HNHYTQKSLSLSPGK
heavy chain constant region with
hole mutations
SEQ ID NO: 19, X1 = S, X2 = A, X3 = V
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSREEMTKNQ
VSLSCAVKGFYPSDIAVEWESNGQPENNYKTTPPV
LDSDGSFFLVSKLTVDKSRWQQGNVFSCSVMHEAL
HNHYTQKSLSLSPGK
light chain constant region
RTVAAPSVFIFPPSDEQLKSGTASVVCLLNNFYPR
EAKVQWKVDNALQSGNSQESVTEQDSKDSTYSLSS
TLTLSKADYEKHKVYACEVTHQGLSSPVTKSFNRG
EC
(SEQ ID NO: 20)
anti-TIGIT heavy chain in MBS310-6
(constant region with hole mutations)
QVQLVQSGAEVKKPGASVKVSCKASGYTFTSYNVH
WVRQAPGQGLEWMGTIYPGNLATSYNQKFKGRVTL
TADTSTSTVYMELSSLRSEDTAVYYCARSGTMDYW
GQGTTVTVSSASTKGPSVFPLAPSSKSTSGGTAAL
GCLVKDYFPEPVTVSWNSGALTSGVHTFPAVLQSS
GLYSLSSVVTVPSSSLGTQTYICNVNHKPSNTKVD
KKVEPKSCDKTHTCPPCPAPELLGGPSVFLFPPKP
KDTLMISRTPEVTCVVVDVSHEDPEVKFNWYVDGV
EVHNAKTKPREEQYNSTYRVVSVLTVLHQDWLNGK
EYKCKVSNKALPAPIEKTISKAKGQPREPQVYTLP
PSREEMTKNQVSLSCAVKGFYPSDIAVEWESNGQP
ENNYKTTPPVLDSDGSFFLVSKLTVDKSRWQQGNV
FSCSVMHEALHNHYTQKSLSLSPGK
(SEQ ID NO: 21)
anti-TIGIT light chain in MBS310-6
EIVLTQSPGTLSLSPGERATMTCRASSSISSTYLH
WYQQKPGASPKLLIYNTQNLASGVPARFSGSGSGT
SYTLTISRLEPEDFAVYYCQQFGGYPLITFGAGTK
LELKRTVAAPSVFIFPPSDEQLKSGTASVVCLLNN
FYPREAKVQWKVDNALQSGNSQESVTEQDSKDSTY
SLSSTLTLSKADYEKHKVYACEVTHQGLSSPVTKS
FNRGEC
(SEQ ID NO: 22)
anti-VEGF heavy chain in MBS310-6
(constant region with knob mutations)
EVQLVESGGGLVQPGGSLRLSCAASGYTFTNYGMN
WVRQAPGKGLEWVGWINTYTGEPTYAADFKRRFTF
SLDTSKSTAYLQMNSLRAEDTAVYYCAKYPHYYGS
SHWYFDVWGQGTLVTVSSASTKGPSVFPLAPSSKS
TSGGTAALGCLVKDYFPEPVTVSWNSGALTSGVHT
FPAVLQSSGLYSLSSVVTVPSSSLGTQTYICNVNH
KPSNTKVDKKVEPKSCDKTHTCPPCPAPELLGGPS
VFLFPPKPKDTLMISRTPEVTCVVVDVSHEDPEVK
FNWYVDGVEVHNAKTKPREEQYNSTYRVVSVLTVL
HQDWLNGKEYKCKVSNKALPAPIEKTISKAKGQPR
EPQVYTLPPSREEMTKNQVSLWCLVKGFYPSDIAV
EWESNGQPENNYKTTPPVLDSDGSFFLYSKLTVDK
SRWQQGNVFSCSVMHEALHNHYTQKSLSLSPGK
(SEQ ID NO: 23)
anti-VEGF light chain in MBS310-6
DIQMTQSPSSLSASVGDRVTITCSASQDISNYLNW
YQQKPGKAPKVLIYFTSSLHSGVPSRFSGSGSGTD
FTLTISSLQPEDFATYYCQQYSTVPWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(SEQ ID NO: 24)
MBS310-7's long chain (anti-VEGF VH-CH-
linker-anti-TIGIT VH-linker-anti-
TIGIT VL)
EVQLVESGGGLVQPGGSLRLSCAASGYTFTNYGMN
WVRQAPGKGLEWVGWINTYTGEPTYAADFKRRFTF
SLDTSKSTAYLQMNSLRAEDTAVYYCAKYPHYYGS
SHWYFDVWGQGTLVTVSSASTKGPSVFPLAPSSKS
TSGGTAALGCLVKDYFPEPVTVSWNSGALTSGVHT
FPAVLQSSGLYSLSSVVTVPSSSLGTQTYICNVNH
KPSNTKVDKKVEPKSCDKTHTCPPCPAPELLGGPS
VFLFPPKPKDTLMISRTPEVTCVVVDVSHEDPEVK
FNWYVDGVEVHNAKTKPREEQYNSTYRVVSVLTVL
HQDWLNGKEYKCKVSNKALPAPIEKTISKAKGQPR
EPQVYTLPPSREEMTKNQVSLTCLVKGFYPSDIAV
EWESNGQPENNYKTTPPVLDSDGSFFLYSKLTVDK
SRWQQGNVFSCSVMHEALHNHYTQKSLSLSPGAGG
GGSGGGGSGGGGSQVQLVQSGAEVKKPGASVKVSC
KASGYTFTSYNVHWVRQAPGQGLEWMGTIYPGNLA
TSYNQKFKGRVTLTADTSTSTVYMELSSLRSEDTA
VYYCARSGTMDYWGQGTTVTVSSGGGGSGGGGSGG
GGSGGGGSEIVLTQSPGTLSLSPGERATMTCRASS
SISSTYLHWYQQKPGASPKLLIYNTQNLASGVPAR
FSGSGSGTSYTLTISRLEPEDEAVYYCQQFGGYPL
ITFGAGTKLTAKR
(SEQ ID NO: 25)
MBS310-7's short chain
(anti-VEGF light chain)
DIQMTQSPSSLSASVGDRVTITCSASQDISNYLNW
YQQKPGKAPKVLIYFTSSLHSGVPSRFSGSGSGTD
FTLTISSLQPEDFATYYCQQYSTVPWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(SEQ ID NO: 24)
MBS310-4's long chain (anti-TIGIT VH-
CH-linker-anti-VEGF VH-linker-anti-
VEGF VL)
QVQLVQSGAEVKKPGASVKVSCKASGYTFTSYNVH
WVRQAPGQGLEWMGTIYPGNLATSYNQKFKGRVTL
TADTSTSTVYMELSSLRSEDTAVYYCARSGTMDYW
GQGTTVTVSSASTKGPSVFPLAPSSKSTSGGTAAL
GCLVKDYFPEPVTVSWNSGALTSGVHTFPAVLQSS
GLYSLSSVVTVPSSSLGTQTYICNVNHKPSNTKVD
KKVEPKSCDKTHTCPPCPAPELLGGPSVFLFPPKP
KDTLMISRTPEVTCVVVDVSHEDPEVKFNWYVDGV
EVHNAKTKPREEQYNSTYRVVSVLTVLHQDWLNGK
EYKCKVSNKALPAPIEKTISKAKGQPREPQVYTLP
PSREEMTKNQVSLTCLVKGFYPSDIAVEWESNGQP
ENNYKTTPPVLDSDGSFFLYSKLTVDKSRWQQGNV
FSCSVMHEALHNHYTQKSLSLSPGAGGGGSGGGGS
GGGGSEVQLVESGGGLVQPGGSLRLSCAASGYTFT
NYGMNWVRQAPGKGLEWVGWINTYTGEPTYAADFK
RRFTFSLDTSKSTAYLQMNSLRAEDTAVYYCAKYP
HYYGSSHWYFDVWGQGTLVTVSSGGGGSGGGGSGG
GGSGGGGSDIQMTQSPSSLSASVGDRVTITCSASQ
DISNYLNWYQQKPGKAPKVLIYFTSSLHSGVPSRF
SGSGSGTDFTLTISSLQPEDEATYYCQQYSTVPWT
FGQGTKLTAKR
(SEQ ID NO: 26)
MBS310-4's short chain
(anti-TIGIT light chain)
EIVLTQSPGTLSLSPGERATMTCRASSSISSTYLH
WYQQKPGASPKLLIYNTQNLASGVPARFSGSGSGT
SYTLTISRLEPEDFAVYYCQQFGGYPLITFGAGTK
LELKRTVAAPSVFIFPPSDEQLKSGTASVVCLLNN
FYPREAKVQWKVDNALQSGNSQESVTEQDSKDSTY
SLSSTLTLSKADYEKHKVYACEVTHQGLSSPVTKS
FNRGEC
(SEQ ID NO: 22)
human TIGIT
MRWCLLLIWAQGLRQAPLASGMMTGTIETTGNISA
EKGGSIILQCHLSSTTAQVTQVNWEQQDQLLAICN
ADLGWHISPSFKDRVAPGPGLGLTLQSLTVNDTGE
YFCIYHTYPDGTYTGRIFLEVLESSVAEHGARFQI
PLLGAMAATLVVICTAVIVVVALTRKKKALRIHSV
EGDLRRKSAGQEEWSPSAPSPPGSCVQAEAAPAGL
CGEQRGEDCAELHDYFNVLSYRSLGNCSFFTETG
(SEQ ID NO: 27)
monkey TIGIT
MAFLVAPPMQFVYLLKTLCVFNMVFAKPGFSETVF
SHRLSFTVLSAVGYFRWQKRPHLLPVSPLGRSMRW
CLFLIWAQGLRQAPLASGMMTGTIETTGNISAKKG
GSVILQCHLSSTMAQVTQVNWEQHDHSLLAIRNAE
LGWHIYPAFKDRVAPGPGLGLTLQSLTMNDTGEYF
CTYHTYPDGTYRGRIFLEVLESSVAEHSARFQIPL
LGAMAMMLVVICIAVIVVVVLARKKKSLRIHSVES
GLQRKSTGQEEQIPSAPSPPGSCVQAEAAPAGLCG
EQQGDDCAELHDYFNVLSYRSLGSCSFFTETG
(SEQ ID NO: 28)
mouse TIGIT
MHGWLLLVWVQGLIQAAFLATGATAGTIDTKRNIS
AEEGGSVILQCHFSSDTAEVTQVDWKQQDQLLAIY
SVDLGWHVASVFSDRVVPGPSLGLTFQSLTMNDTG
EYFCTYHTYPGGIYKGRIFLKVQESSVAQFQTAPL
GGTMAAVLGLICLMVTGVTVLARKKSIRMHSIESG
LGRTEAEPQEWNLRSLSSPGSPVQTQTAPAGPCGE
QAEDDYADPQEYFNVLSYRSLESFIAVSKTG
(SEQ ID NO: 29)
human PVR
MARAMAAAWPLLLVALLVLSWPPPGTGDVVVQAPT
QVPGFLGDSVTLPCYLQVPNMEVTHVSQLTWARHG
ESGSMAVFHQTQGPSYSESKRLEFVAARLGAELRN
ASLRMFGLRVEDEGNYTCLFVTFPQGSRSVDIWLR
VLAKPQNTAEVQKVQLTGEPVPMARCVSTGGRPPA
QITWHSDLGGMPNTSQVPGFLSGTVTVTSLWILVP
SSQVDGKNVTCKVEHESFEKPQLLTVNLTVYYPPE
VSISGYDNNWYLGQNEATLTCDARSNPEPTGYNWS
TTMGPLPPFAVAQGAQLLIRPVDKPINTTLICNVT
NALGARQAELTVQVKEGPPSEHSGISRNAIIFLVL
GILVFLILLGIGIYFYWSKCSREVLWHCHLCPSST
EHASASANGHVSYSAVSRENSSSQDPQTEGTR
(SEQ ID NO: 30)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A bispecific molecule, comprising a TIGIT binding domain and a VEGF binding domain, wherein the TIGIT binding domain comprises an anti-TIGIT antibody or an antigen binding fragment thereof, wherein the VEGF binding domain comprises an anti-VEGF antibody or an antigen binding fragment thereof.

2. The bispecific molecule of claim 1, comprising:
i) a first polypeptide, containing, from N-terminus to C-terminus, an anti-TIGIT heavy chain variable region and a heavy chain constant region,
ii) a second polypeptide, containing an anti-TIGIT light chain variable region,
iii) a third polypeptide, containing, from N-terminus to C-terminus, an anti-VEGF heavy chain variable region, and a heavy chain constant region, and
iv) a fourth polypeptide, containing an anti-VEGF light chain variable region,
wherein the anti-TIGIT heavy chain variable region in the first polypeptide and the anti-TIGIT light chain variable region in the second polypeptide associate to form the anti-TIGIT binding domain, the anti-VEGF heavy chain variable region in the third polypeptide and the anti-VEGF light chain variable region in the fourth polypeptide associate to form the anti-VEGF binding domain, and the heavy chain constant region in the first polypeptide and the heavy chain constant region in the third polypeptide are associated together; or
i) a first polypeptide, containing an anti-VEGF heavy chain variable region, a heavy chain constant region, an anti-TIGIT heavy chain variable region and an anti-TIGIT light chain variable region,
ii) a second polypeptide, containing an anti-VEGF light chain variable region,
iii) a third polypeptide, containing an anti-VEGF heavy chain variable region, a heavy chain constant region, an anti-TIGIT heavy chain variable region and an anti-TIGIT light chain variable region, and
iv) a fourth polypeptide, containing an anti-VEGF light chain variable region,
wherein the anti-VEGF heavy chain variable region in the first polypeptide and the anti-VEGF light chain variable region in the second polypeptide associate to form the VEGF binding domain, the anti-TIGIT heavy chain variable region and the anti-TIGIT light chain variable region in the first polypeptide associate to form the TIGIT binding domain, the anti-VEGF heavy chain variable region in the third polypeptide and the anti-VEGF light chain variable region in the fourth polypeptide associate to form the VEGF binding domain, the anti-TIGIT heavy chain variable region and the anti-TIGIT light chain variable region in the third polypeptide associate to form the TIGIT binding domain, and the heavy chain constant region in the first polypeptide and the heavy chain constant region in the third polypeptide are associated together.

3. The bispecific molecule of claim 2, which is afucosylated.

4. The bispecific molecule of claim 3, wherein the heavy chain constant region in the first polypeptide which comprises, from N-terminus to C-terminus, the anti-TIGIT heavy chain variable region and the heavy chain constant region, is with hole mutation(s), and the heavy chain constant region in the third polypeptide which comprises, from N-terminus to C-terminus, the anti-VEGF heavy chain variable region and the heavy chain constant region, is with knob mutation(s); or
the heavy chain constant region in the first polypeptide which comprises, from N-terminus to C-terminus, the anti-TIGIT heavy chain variable region and the heavy chain constant region, is with knob mutation(s), and the heavy chain constant region in the third polypeptide which comprises, from N-terminus to C-terminus, the anti-VEGF heavy chain variable region and the heavy chain constant region, is with hole mutation(s).

5. The bispecific molecule of claim 4, wherein the second polypeptide further comprises a light chain constant region at the C-terminus, and/or the fourth polypeptide further comprises a light chain constant region at the C-terminus.

6. The bispecific molecule of claim 2, wherein the anti-VEGF heavy chain variable region comprises a VH-CDR1, a VH-CDR2 and a VH-CDR3 comprising the amino acid sequences of SEQ ID NOs: 7, 8 and 9, respectively, and the anti-VEGF light chain variable region comprises a VL-CDR1, a VL-CDR2 and a VL-CDR3 comprising the amino acid sequences of SEQ ID NOs: 10, 11 and 12, respectively.

7. The bispecific molecule of claim 6, wherein the anti-VEGF heavy chain variable region and the anti-VEGF light chain variable region comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 15 and 16, respectively.

8. The bispecific molecule of claim 7, wherein the anti-TIGIT heavy chain variable region comprises a VH-CDR1, a VH-CDR2 and a VH-CDR3 comprising the amino acid sequences of SEQ ID NOs: 1, 2 and 3, respectively, and the anti-TIGIT light chain variable region comprises a VL-CDR1, a VL-CDR2 and a VL-CDR3 comprising the amino acid sequences of SEQ ID NOs: 4, 5 and 6, respectively.

9. The bispecific molecule of claim 8, wherein the anti-TIGIT heavy chain variable region and the anti-TIGIT light chain variable region comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 13 and 14, respectively.

10. The bispecific molecule of claim 9, wherein the heavy chain constant region in the first polypeptide which comprises, from N-terminus to C-terminus, the anti-TIGIT heavy chain variable region and the heavy chain constant region, comprises the amino acid sequence of SEQ ID NO: 19 (X1=s, X2=A, X3=V), and the heavy chain constant region in the third polypeptide which comprises, from N-terminus to C-terminus, the anti-VEGF heavy chain variable region and the heavy chain constant region, comprises the amino acid sequence of SEQ ID NO: 19 (X1=W, X2=L, X3=Y).

11. The bispecific molecule of claim 9, wherein the first, second, third and fourth polypeptides comprise the amino acid sequences of i) SEQ ID NOs: 21, 14, 23 and 16, respectively; ii) SEQ ID NOs: 21, 22, 23 and 24, respectively; iii) SEQ ID NOs: 25, 16, 25 and 16, respectively; or iv) SEQ ID NOs: 25, 24, 25 and 24, respectively.

12. A nucleic acid molecule, encoding the bispecific molecule of claim 1.

13. An expression vector comprising the nucleic acid molecule of claim 12.

14. A host cell comprising the expression vector of claim 13.

15. A pharmaceutical composition comprising the bispecific molecule of claim 1 and a pharmaceutically acceptable carrier.

16. A method for treating or alleviating a tumor associated with TIGIT signaling and/or VEGF signaling in a subject in need thereof, comprising administering to the subject a therapeutically effective amount of the pharmaceutical composition of claim 15.

17. The method of claim 16, wherein the tumor is a solid tumor.

18. The method of claim 17, wherein the tumor is colorectal cancer, liver cancer, endometrial cancer, pancreatic cancer, non-small-cell carcinoma, multiple myeloma, melanoma, renal cell carcinoma, glioblastoma multiforme, ovarian cancer, hepatocellular carcinoma, or cervical carcinoma.

19. A method for treating or alleviating a neovascular eye disease associated with TIGIT signaling and/or VEGF signaling in a subject in need thereof, comprising administering to the subject a therapeutically effective amount of the pharmaceutical composition of claim 15.

20. The method of claim 19, wherein the neovascular eye disease is diabetic macular edema, diabetic retinopathy, retinal vein occlusion, age-related macular degeneration, or choroidal neovascularization.
